(19) United States
(12) Patent Application Publication
(10) Pub. No.: US 2020/0002749 A1
(43) Pub. Date: Jan. 2, 2020

(54) METHOD AND SYSTEM FOR DIFFERENTIATING MORE PATHOGENIC STAPHYLOCOCCUS STRAINS

(71) Applicant: University of Iowa Research Foundation, Iowa City, IA (US)

(72) Inventor: Randy W. Loftus, Iowa City, IA (US)

(21) Appl. No.: 16/434,652

(22) Filed: Jun. 7, 2019

Related U.S. Application Data
(60) Provisional application No. 62/682,267, filed on Jun. 8, 2018.

(51) Int. Cl. C12Q 1/689; C12Q 1/686; C12Q 1/6869; C12Q 1/6858; C12Q 1/6827

(52) U.S. Cl. CPC C12Q 1/689; C12Q 1/686; C12Q 1/6827; C12Q 1/6858; C12Q 1/6869

(57) ABSTRACT

The disclosure provides for a method and system for differentiation of more pathogenic bacterial strains among commonly isolated intraoperative multilocus sequence types.

### CROSS REFERENCE TO RELATED APPLICATIONS

This application claims the benefit of the filing date of U.S. application No. 62/682,267, filed on Jun. 8, 2018, the disclosure of which is incorporated by reference herein.

### FIELD OF THE INVENTION

The invention relates to methods and systems for identifying certain infections in patients. In particular, a panel in a system that can be used to differentiate more pathogenic S. aureus strains among commonly isolated intraoperative multilocus sequence types.

### BACKGROUND

Healthcare-associated infections (HAIs) affect up to 7% of patients undergoing surgery. National organizations such as the Centers for Disease Control (CDC) and the World Health Organization (WHO) consider HAIs to be a devastating and persistent problem linked to antibiotic resistance and significant increases in healthcare costs. The CDC has highlighted three major goals for HAI prevention including the following: 1) prevention of infections in patients undergoing surgery, 2) prevention of patient-to-patient bacterial transmission, and 3) improvement in antibiotic stewardship.

The contribution of intraoperative bacterial reservoirs to bacterial transmission events and postoperative infection development has been characterized. In one study, bacterial contamination of the anesthesia environment increased significantly during the administration of general anesthesia and was associated with an increased risk of patient intravenous stopcock set contamination. In turn, stopcock contamination was linked by pulsed-field gel electrophoresis (PFGE) to postoperative infection development and was associated with increased patient mortality. In a subsequent multicenter study, stopcock contamination was detected in 23% of surgical cases, associated with increased mortality, and again linked by PFGE to postoperative infection. Intraoperative bacterial reservoir isolates were directly linked by PFGE to the causative organism of infection for 30% of 30-day postoperative HAIs.

S. aureus transmission has been confirmed in up to 39% of surgical cases involving all-comers to the operating room and linked to postoperative infection development. As S. aureus is a frequently transmitted intraoperative pathogen and a leading cause of blood stream and respiratory infections, attenuation of intraoperative S. aureus transmission is an important target for HAI prevention. Previous attempts to address related problems have included PCR-based diagnostic for detection of methicillin sensitive and resistant S. aureus (MSSA and MRSA, respectively). However, these diagnostics have not been shown to be particularly useful in preventing SSIs and are in need of advancement.

### SUMMARY

The disclosure provides for systems and methods of identifying bacterial, for example, Staphylococcus, e.g., S. aureus, transmissions and/or infections. The use of the systems and methods may allow for prevention and/or inhibition of further transmission of S. aureus transmissions.

In one embodiment, an allelic discrimination panel is provided. The panel includes a multilocus sequence type (MLST) assay, wherein the MLST assay detects a bacterial genus, bacterial species, a bacterial strain, or a combination thereof. In one embodiment, the MLST assay comprises a polymerase chain reaction (PCR) platform, a PCR seal, a PCR, a primer, a PCR reagent, a probe, or combination thereof. In one embodiment, the PCR is a real-time PCR. In one embodiment, the MLST assay is an S. aureus assay, which detects an S. aureus strain. In one embodiment, the MLST assay is an MLST 5, MLST 8, MLST 15, MLST 30, or MLST 59 assay of S. aureus. In one embodiment, the MLST assay targets a position in a bacteria genome, a position in a bacterial plasmid, or a combination thereof. In one embodiment, the position in the bacteria genome and/or the position in the bacterial plasmid is one or more of the following 189723, 716410, 1517201, 1517381, 64695, 64954, 64974, 65001, 65096, 65216, 66001, 66200, 66543, 105870, 657832, 727126, 908100, 1186436, 1419668, 1656006, 1762538, 1897263, 2209374, 2211044, or 2257298. In one embodiment, the panel includes another assay wherein the additional assay detects a bacterial genus and/or species. In one embodiment, the second MLST assay detects an additional sequence type. In one embodiment, the probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a combination thereof.

As also described below, S. aureus ST 5 was associated with increased risk of transmission (IRR adj 6.67, 95% CI 1.82-24.41, P=0.0008), greater biofilm absorbance [(ST 5 median absorbance 3.08, SD 0.642) vs. (other ST median absorbance 2.38, SD 1.01), corrected P=0.021], multidrug resistance (OR 7.82, 95% CI 2.19-27.95, P=0.002), and infection (6/38 ST 5 vs. 6/140, RR 3.68, 95% CI 1.26-10.78, P=0.022). Provider hands (N=3) and patients (N=4) were confirmed sources of ST 5 transmission. Transmission locations included provider hands (N=3), patient skin sites (N=4), and environmental surfaces (N=2). All observed transmission stories involved the within-case mode of transmission. Two of the sequence type 5 transmission events were directly linked to infection. Thus, intraoperative S. aureus ST 5 isolates are hyper transmissible and pathogenic.

Desiccation tolerance increases Staphylococcus aureus survival and risk of transmission. As described below, S. aureus isolates (N=173) were collected from anesthesia work area reservoirs in 55 operating room environments, and desiccation tolerance was assessed. The association of increased desiccation tolerance with S. aureus sequence type (ST), clonal transmission, and the spread of mecA and methicillin-resistance was evaluated. Whole cell genome analysis was used to compare desiccation tolerant isolates to causative organisms of infection. S. aureus ST 5 isolates had greater desiccation tolerance as compared to all other ST [(ST 5, N=34, median CFU/ml 136,000), (other ST, N=139, median CFU/ml=42,200), corrected P<0.0001] in this study. ST 5 was associated with increased risk of clonal transmission (RR 1.81, 95% CI 1.22-2.69, p=0.003). Transmitted ST 5 isolates were associated with the mecA resistance trait (adjusted OR 14.81, 95% CI 3.83-57.19, P=0.0001) and methicillin-resistance (adjusted OR 4.25, 95% CI 1.29-13.98, P=0.02). Two desiccation tolerant ST 5 isolates were linked to infection. Thus, intraoperative S. aureus ST 5 (USAlOO) is related to enhanced desiccation tolerance, increased transmission, and directly linked to postoperative infection.

Further provided is a method of identifying and/or monitoring a pathogenic bacterial strain among commonly isolated intraoperative multilocus sequence types in a mammal, e.g., a human. The method includes performing an MLST assay comprising detecting the presence of an MLST of a bacteria, wherein the detecting is performed by a real-time PCR comprising a probe. In one embodiment, the MLST assay is an S. aureus MLST assay. In one embodiment, the MLST assay is an MLST 5, MLST 8, MLST 15, MLST 30, or MLST 59 assay of S. aureus. In one embodiment, the MLST assay PCR probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a combination thereof. In one embodiment, the MLST assay targets a position in a bacteria genome, a position in a bacterial plasmid, or a combination thereof. In one embodiment, the position in the bacteria genome and/or the position in the bacterial plasmid is one or more of the following 189723, 716410, 1517201, 1517381, 64695, 64954, 64974, 65001, 65096, 65216, 66001, 66200, 66543, 105870, 657832, 727126, 908100, 1186436, 1419668, 1656006, 1762538, 1897263, 2209374, 2211044, and 2257298.

Further provided is a kit for identifying and/or monitoring pathogenic bacterial strains among commonly isolated intraoperative multilocus sequence types which includes the allelic discrimination panel. In one embodiment, the kit further comprises instructions, a swab, a buffer, a glove, or a combination thereof.

### BRIEF DESCRIPTION OF THE FIGURES

FIG. 1. Allelic discrimination plot.

FIG. 2. Allelic discrimination plot.

FIG. 3. Allelic discrimination plot.

FIG. 4. Allelic discrimination plot.

FIG. 5. Allelic discrimination plot.

FIG. 6. Allelic discrimination plot.

FIG. 7. Allelic discrimination plot.

FIG. 8. Allelic discrimination plot.

FIG. 9. Conventional Lab Processing Versus Rapid Diagnostic Testing.

### DETAILED DESCRIPTION

The present disclosure relates to methods and systems for identifying bacteria involved in HAIs, such as the more pathogenic S. aureus strains among commonly isolated intraoperative multilocus sequence types. The methods and systems described herein have many advantages over existing methods and systems for identifying bacterial infections and transmissions. For example, the methods and systems can differentiate and identify more pathogenic, intraoperative S. aureus multilocus sequence types (MLST) defined by strong biofilm formation and multidrug-resistance (MDR) and to characterize the dynamics of their intraoperative spread. Furthermore, the systems and methods are capable of faster, e.g., rapid, identification of more pathogenic bacteria, such as S. aureus, infection and transmission.

The embodiments of this disclosure are not limited to particular infection monitoring systems, which can vary and are understood by skilled artisans. It is further to be understood that all terminology used herein is to describing particular embodiments only, and is not intended to be limiting in any manner or scope. So that the present disclosure may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

### Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the certain materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

The term “about,” as used herein, refers to variation in the numerical quantity that can occur, for example, through typical measuring techniques and equipment, with respect to any quantifiable variable, including, but not limited to, mass, volume, concentration, and time. Whether or not modified by the term “about,” the claims include equivalents to the quantities.

The term “MLST” as used herein refers to a multilocus sequence type, which can be used interchangeably with strain.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides. In one embodiment, for diagnostic applications, depending on the complexity of the target sequence, the oligonucleotide probe may contain 10, 15, 126, 17, 18, 19, 20, 21, 22, 23, 24 25, 26, 27, 28 29 or 30 or more nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. In one embodiment, the probe for a highly transmissible S. aureus has at least 80%, 82%, 85%, 87%, 90%, 92%, 95%, 98% or 99% identity to one of SEQ ID Nos. 1-8, 13-14, 17-18, 21-22, or 25-26. In one embodiment, the probe for a highly transmissible S. aureus has at least 1, 2, 3, 4 or 5 nucleotide substitutions relative to one of SEQ ID Nos. 1-8, 13-14, 17-18, 21-22, or 25-26.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. In one embodiment, for diagnostic applications, the oligonucleotide primer may contain 10, 11, 12, 13, 14, 15, 126, 17, 18, 19, 20, 21, 22, 23, 24 25, 26, 27, 28 29 or 30 or more nucleotides. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product. In one embodiment, a primer to amplify nucleic acid from a highly transmissible S. aureus has at least 80%, 82%, 85%, 87%, 90%, 92%, 95%, 98% or 99% identity to one of SEQ ID Nos. 15-16, 19-20, 23-24 or 27-28. In one embodiment, a primer to amplify nucleic acid from a highly transmissible S. aureus has at least 1, 2, 3, 4 or 5 nucleotide substitutions relative to one of SEQ ID Nos. 15-16, 19-20, 23-24 or 27-28.

Polymerase chain reaction (PCR) has been described in U.S. Pat. Nos. 4,683,195, 4,800,195, and 4,965,188, the entire disclosures of which are incorporated by reference herein.

As used herein, the terms “reporter,” “reporter system,” “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The term “sample,” as used herein, refers to a bacterial sample taken from a patient, attending medical personnel skin, clothing, or gloves, or the environment, such as, but not limited to, an operating room, operating equipment, waiting rooms, bathrooms, and/or patient rooms. “Sample” may be used interchangeable with the term “lysate.”

The term “methicillin-resistant Staphylococcus aureus,” “MRSA,” as used herein, refers to a strain of S. aureus which is resistant to methicillin. The term “methicillin-susceptible Staphylococcus aureus,” “MSSA,” as used herein, refers to a strain of S. aureus which may be treated by methicillin.

The methods and compositions of the present invention may comprise, consist essentially of, or consist of the components and ingredients of the present invention as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

### Allelic Discrimination Panel

One embodiment is an allelic discrimination panel. In one embodiment, the allelic discrimination panel can be employed in a method of identifying a pathogenic bacterial strain. In one embodiment, the allelic discrimination panel can be included in a kit. A kit comprising an allelic discrimination panel can further comprise instructions, a swab, a buffer, a glove, or a combination thereof. The instructions can be provided online, in a print copy, or both.

Allelic discrimination panels can be made for any bacterial strain. The panels employ one or more assays to identify genotypes. The genotypes can then quickly be linked to various phenotypic analysis.

The panel may comprise a real time polymerase chain reaction (PCR) platform, a PCR seal, PCR primers, PCR reagents, and one or more probes. The panel may also include high throughput sequencing or PCR followed by restriction fragment length polymorphism using methods known in the art.

By way of example, the PCR platform may comprise, but not limited to, a 96-well or 48-well plate, or a 384-well card, and may have a full, partial or no skirt. The PCR platform may be optically active for real time polymerase chain reaction. The PCR seal may be optically clear film and may have one side comprised of an adhesive used to apply the seal to the top of the plate.

The PCR primers may consist of sequences between 6 and 50 nucleotides long which may flank one or more loci of interest in the bacterial genome. The PCR reagents may comprise water, PCR buffer, magnesium chloride, magnesium sulfate, a DNA polymerase, deoxy-nucleotide tri-phosphates, or dimethyl sulfoxide. The DNA polymerase may be any type, including low fidelity, such as but not limited to Taq, to high fidelity, such as but not limited to Pfx.

The PCR probe may comprise of a reporter and a quencher, such as but not limited to TAQMAN, and may be positioned such that when the DNA polymerase read the bacterial genome, the reporter may be freed from the quencher into solution. If multiple probes are used in parallel, each probe will have its own reporter. Reporters include, but are not limited to, FAM, JOE, Texas Red, Cy®3, TAMRA, or Cy®5. Passive dyes may also be used and include, but are not limited to, ROX™ or MUSTANG PURPLE™.

The probes and primers may be added to each well of the assay and then dried for quick use, added to a master mix prior to loading the master mix, or added individually to the well. All probes and primers should be selected to run together for the same annealing temperature if ran on the same plate or card. Optionally, depending on the system, a gradient of annealing temperatures may be run across the plate or card.

Each well of the assay may be loaded with a master mix and the either a sample or a control. The master mix may comprise of DNase and nuclease free water, buffer, deoxynucleic acid tri-phosphate mix (dNTP), a magnesium source, a polymerase, and a passive reference dye. The master mix may further contain primers, probes, samples, or controls. The control may be a no DNA control or a negative DNA control comprising of the DNA from a different bacterial type.

Next generation sequencing can be performed by multiple methods, including, but not limited to, single-molecule real-time sequencing, ion semiconductor, pyrosequencing, sequencing by ligation, nanopore, or chain termination. Each next generation sequencing method has its own procedure known in the art. Prepping the DNA may be done through first isolating the DNA using techniques well known in the art. The isolated DNA may then be shortened to the appropriate length for sequencing such as by using sonication, enzymatic shearing. Libraries were then made by performing end repair, A-tailing, ligation, and amplification.

More than one Panel may be used to detect different alleles, such as, but not limited to, a two-panel system which a first panel is used to detect common isolates and a second panel to detect specific isolates. Additional panels may be used to test for different, specific isolates, creating systems with three or more Panels.

The Panels may be used to detect every locus of a multilocus sequence type. Alternatively, a representative allele unique to each multilocus sequence type may be assayed. A combination, in which some multilocus sequence types are represented by more than one locus on the assay in the Panel and some multilocus sequence types are represented by a single locus on the assay.

In one embodiment, an allelic discrimination panel can be selected to target a particular MLST, a particular position in a bacteria genome, a particular position in a bacterial plasmid, or a combination thereof. In one embodiment, the MLST targeted include, but are not limited to, an MLST of S. aureus. In one embodiment, S. aureus MLSTs include, but are not limited to, MLST 5, MLST 8, MLST 15, MLST 30, MLST 59, or a combination thereof. In an aspect of the invention, particular positions and MLSTs can be detected by use of a probe which detects a particular allele. Table 1 below provides a list of exemplary positions. Table 2 below provides a list of exemplary probes.

TABLE 1
Allelic Discriminatory Panel Assayed Genomic Positions

| Organism | Position | Type | Reference | Allele | MLST |
|---|---|---|---|---|---|
| S. aureus | 189723 | SNP | G | C | 5 |
| S. aureus | 716410 | SNP | C | T | 5 |
| S. aureus | 1517201 | SNP | G | A | 5 |
| S. aureus | 1517381 | SNP | G | A | 5 |
| S. aureus | 64695 | SNP | A | T | 5/8 |
| S. aureus | 64954 | SNP | G | A | 5/8 |
| S. aureus | 64974 | SNP | T | G | 5/8 |
| S. aureus | 65001 | SNP | C | T | 5/8 |
| S. aureus | 65096 | SNP | T | G | 5/8 |
| S. aureus | 65216 | SNP | T | C | 5/8 |
| S. aureus | 66001 | SNP | C | T | 5/8 |
| S. aureus | 66200 | SNP | T | C | 5/8 |
| S. aureus | 66543 | SNP | A | G | 5/8 |
| S. aureus | 105870 | SNP | G | C | |
| S. aureus | 657832 | SNP | G | A | |
| S. aureus | 727126 | SNP | T | C | |
| S. aureus | 908100 | SNP | G | A | |
| S. aureus | 1186436 | SNP | T | G | |
| S. aureus | 1419668 | SNP | T | G | |
| S. aureus | 1656006 | Deletion | G | — | |
| S. aureus | 1762538 | SNP | A | G | |
| S. aureus | 1897263 | SNP | A | G | |
| S. aureus | 2209374 | SNP | A | G | |
| S. aureus | 2211044\hat{ }2211045 | Insertion | — | GCA | |
| S. aureus | 2257298 | SNP | G | A | |

TABLE 2

| SEQ ID NO. | Organism | Probe | Sequence | Target |
|---|---|---|---|---|
| SEQ ID NO: 1 | S. aureus | Probe 1 | AACATTGTAGC | MLST5 |
| | | | GCCTAA | |
| SEQ ID NO: 2 | S. aureus | Probe 2 | AAACATTGTAC | MLST5 |
| | | | CGCCTAA | |
| SEQ ID NO: 3 | S. aureus | Probe 1 | AAGAAAGAAAA | MLST5 |
| | | | CAAGCGCTA | |
| SEQ ID NO: 4 | S. aureus | Probe 2 | AAGAAAGAAAA | MLST5 |
| | | | CAAGCGTTA | |
| SEQ ID NO: 5 | S. aureus | Probe 1 | TGTTCAACGGC | MLST5 |
| | | | TGCT | |
| SEQ ID NO: 6 | S. aureus | Probe 2 | TGTTCAACGGC | MLST5 |
| | | | TACTT | |
| SEQ ID NO: 7 | S. aureus | Probe 1 | TGTATTTAATT | MLST5 |
| | | | CAGATGCCGTT | |
| SEQ ID NO: 8 | S. aureus | Probe 2 | ATTTAATTCAG | MLST5 |
| | | | ATGCCATTG | |

### Sample Preparation

Samples may be obtained from by swabbing any surface including, but not limited to, from a patient's skin or clothing, attending medical personnel's skin, clothing, or gloves, or the environment, such as, but not limited to, an operating room, operating equipment, waiting room, bathroom, patient room, ambulance, or combination thereof. Swabs may be taken using any absorbent material such as, but not limited to, cotton, polyester, such as rayon or dacron, or charcoal. Swabs may form an absorbent foam or be flocked.

Swabbing may occur in route to the hospital in an ambulance, just prior to surgery, or days in advance of surgery depending on of the surgery is planned or an emergency.

Depending on the expected number of bacteria obtained on the swab, the sample may be placed into a container, such as a conical glass tube or microcentrifugal, with collection buffer such as, but not limited to Aimes Transport Medium. The swab may also be placed into a growth media and incubated to boost the bacterial count. After either transport or incubation, the sample may then be lysed or have phenotypic analysis performed. Lysing methods are known in the art and include, but are not limited to chemical methods, such as salt and/or detergent treatment, freeze/thaw cycling, sonication, microbead disruption, and/or boiling.

The lysed samples may then be run on one or more Allelic Discriminating Panels, such as but not limited to the one described in the above Examples and/or a MRSA and/or MSSA tests, to identify possible intraoperative bacteria and possible antimicrobial resistances, such as but not limited to S. aureus.

### Phenotypic Analysis

Phenotypic analysis includes bacterial traits related to virulence, such as, but not limited to, antibiotic resistance, desiccation tolerance, and/or biofilm formation. Optionally, phenotypic data may be based on any known mutation relating to a trait, such as, but not limited to, SarA and Agr being related to biofilm formation and desiccation tolerance.

Antibiotic resistance may be assayed by any technique known in the art such as, but not limited to, broth microdilution, rapid automated susceptibility testing methods, disk or gradient diffusion, E-test, mechanism-specific tests, such as, but not limited to, beta-lactamase detection and chromogenic cephalosporin test, genotyping, or direct single-cell imaging.

Any antibiotic known in the art may be tested, including, but not limited to, aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin. A bacterium may be assayed for one or more antibacterial agent.

Desiccation tolerance may be assayed by any technique known in the art such as, but not limited to, drying followed by rehydration. Drying may be performed with or without a chemical desiccant, drying with or without a vacuum, or using an aerogel or molecular sieve. A chemical desiccant may include, but is not limited to, activated alumina, benzophenone, bentonite clay, calcium chloride, calcium oxide, calcium sulfate, cobalt(II) chloride, copper(II) sulfate, lithium chloride, lithium bromide, magnesium sulfate, magnesium perchlorate, potassium carbonate, potassium hydroxide, silica gel, sodium, sodium chlorate, sodium chloride, sodium hydroxide, sodium sulfate, sucrose, or sulfuric acid, or combinations thereof. Aerogels may be made by any technique, such as, but not limited to, sol-gel or pyrolysis, or compound known to the art. Compounds include, but are not limited to, silica, carbon, metal oxides, agar, cellulose, chalcogens, metals, and nanoparticles, or combinations thereof. Aerogels may also include dopants or reinforcing compounds such as, but not limited to, fiberglass. Molecular sieves may comprise of zeolites, aluminum oxides, porous glass, active carbon, slays, silicon dioxide, silica, metal oxides, or combinations thereof. Rehydration may be done with sterile water, with or without a buffer, or growth media.

Biofilm formation may be assayed by any device and/or technique known to the art. Devices include microtiter plates, Robbins devices, drip flow biofilm reactor, rotary biofilm reactors, direct inspection, and devices biofilm microfluidic devices. Rotary biofilm reactors include rotary annular reactors, rotary disk reactors, and concentric cylinder reactors. Direct inspection devices include open channel flat plate reactors and closed flow channels. Methods to measure aspects of the biofilm include adhesion strength, adhesion extent and biofilm biomass and viability, and biofilm matrix composition. Adhesion strength can be measured using AFM, QCM, and/or SPR. Adhesion extent and biofilm biomass and viability can be either directly or indirectly measured. Direct measurements include light microscopy, epifluorescence, CLSM, SEM, Cryo-SEM, e-SEM, Bif-SEM, and/or AFM. Indirect measures include cell viability, cellular biomass, metabolic activity, and/or biofilm biomass. Examples of cell viability include CFU, PMA-qPCR, flow-cytometry, and/or phospholipid-analysis. Examples of cellular biomass assays include qPCR and flow-cytometry. Examples of metabolic activity assays include dye staining, include XTT, TTC, resazurin alamar blue. Biofilm biomass assays include dye staining, such as crystal violet, weight, EIS, and UTDR. Biofilm matrix composition can include in situ or ex situ methods. Examples of in situ methods include CLSM and FCS. Examples of ex situ methods include matrix extraction, using either physical or chemical methods, and analytical methods to measure EPS.

Genotyping can be done using any method known in the art. Examples include, but are not limited to, restriction fragment length polymorphism identification of genomic DNA, random amplified polymorphic detection of genomic DNA, amplified fragment length polymorphism detection, polymerase chain reaction, DNA sequence, such as Sanger sequences or high-throughput sequencing, allele specific oligonucleotide probes, and hybridization techniques, such as hybridization to DNA microarrays or beads. Bacteria may be genotyped at a single locus or at multiple loci.

### Results

The discrimination assay results can be processed by a custom laboratory information management system. An exemplary custom laboratory information management system is available from OR PathTrac, RDB Bioinformatics, Omaha, Nebr. 68154, which is described in PCT/US17/26557, which is expressly incorporated herein in its entirety. The laboratory information management system can guide improved perioperative infection control measures based on enhanced detection and surveillance of these key strain characteristics identified in the allelic panel. This discrimination panel working in conjunction with a laboratory information management system allows acute care settings to respond to high-risk exposure with an advanced, evidence-based, multimodal program designed to optimize perioperative patient safety. Through continual surveillance as provide by laboratory information management system component, such as OR PathTrac, the fidelity of bundle components can be measured individually and collectively.

### Treatment

If the one or more Allelic Discriminating Panels have a positive result for bacteria related to healthcare-related infections, the patient may then be treated with an appropriate treatment. Exemplary treatments, include, but are not limited to, chlorhexidine, vancomycin, cefazolin, and nasal mupirocin or povidone iodine if the patient is not allergic. Treatment may last between two and five days prior to surgery. The patient's skin may be also treated just prior to surgery with alcohol and chlorhexidine to create an aseptic cutting area.

In an embodiment of the invention, a kit comprises one or more Allelic Discrimination Panels comprises two real-time PCR based assays, one to detect common bacterial species and/or genera, the other to detect specific multilocus sequence type strains and instructions. A further embodiment is a S. aureus Allelic Discrimination Panel. In a further embodiment, one Allelic Discrimination Panel tests for S. aureus common isolates and another Panel tests for the multilocus sequence type 5 of S. aureus. In yet a further embodiment, the kit contains swabs and transfer buffer. In a still further embodiment, the kit contains a lysis buffer.

In another embodiment, the one or more Allelic Discrimination Panels test for Enterococcus. In a further embodiment, the Allelic Discrimination Panel tests for Enterococcus multilocus sequence types E5 and E7.

In another embodiment, one or more Allelic Discrimination Panels are run in parallel. In another embodiment, the Panels are run sequentially. In a further embodiment, the panels are executed in parallel preoperatively, before surgery. Clinical application of the panel involves the addition of assays 1 and 2 to conventional, preoperative MSSA and MRSA testing. MRSA/MSSA identifies whether S. aureus is present or not and provides rapid insight into antibiotic susceptibility in order that prophylactic antibiotics can be tailored.

In another embodiment of the invention, patients arriving at the Emergency Room of a hospital to undergo urgent treatment will receive nasal povidone iodine and chlorhexidine wipe decontamination prior to surgery and on postoperative day 1 as the patient does not have time to undergo the longer treatment. The patient may be swabbed in the waiting room by a nurse or orderly, and the swab may then be sent for lysis and testing on one or more Allelic Discriminating Panels.

In another embodiment of the invention, patients arriving at the hospital by emergency vehicle, such as but not limited to helicopter or ambulance, may be swabbed in route. Upon the patient arrival to the hospital, the swab may then be sent to be lysed and analyzed.

In another embodiment, environmental samples can be taken from equipment in an operating room, tools used during surgery, the walls of an operating room, the patient's room, high traffic areas of a hospital, the emergency room waiting area, and/or inside emergency vehicles.

In another embodiment, samples from attending health care workers can be taken from their skin, cloths, or gloves before or after attending to a patient.

In another embodiment, the Allelic Discrimination Panel comprises a whole cell genome analysis, next generation sequencing.

Table 3, below, provides the sequences of exemplary primers, probes, and their flanking sequences.

TABLE 3

| Name | Organism | Primer/Probe | Sequence | Target |
|---|---|---|---|---|
| 053-probe-100 | S. aureus | VIC PROBE | TACGTTTGAACTCTGCGAC | |
| | | | (SEQ ID NO: 9) | |
| 053-probe-100 | S. aureus | FAM PROBE | CGTTTGAACTCTGCAAC | |
| | | | (SEQ ID NO: 10) | |
| 053-probe-100 | S. aureus | Forward P | CCTTCAAGTTTAGCACTAC | |
| | | | GACCTT (SEQ ID NO: 11) | |
| 053-probe-100 | S. aureus | Reverse P | GCAAACGGACATAAAGTA | |
| | | | GTAACTGAAA (SEQ ID | |
| | | | NO: 12) | |
| MST5 | S. aureus | Probe 1 | AACATTGTAGCGCCTAA | MLST5 |
| | | | (SEQ ID NO: 13) | |
| MST5 | S. aureus | Probe 2 | AAACATTGTACCGCCTAA | MLST5 |
| | | | (SEQ ID NO: 14) | |
| MST5 | S. aureus | Forward P | GAGCCATACTTCGACAAA | MLST5 |
| | | | CTAACATAA (SEQ ID | |
| | | | NO: 15) | |
| MST5 | S. aureus | Reverse P | TGCGCCTATGACATTGATT | MLST5 |
| | | | AATG (SEQ ID NO: 16) | |
| MST5 | S. aureus | Probe 1 | AAGAAAGAAAACAAGCGC | MLST5 |
| | | | TA (SEQ ID NO: 17) | |
| MST5 | S. aureus | Probe 2 | AAGAAAGAAAACAAGCGT | MLST5 |
| | | | TA (SEQ ID NO: 18) | |
| MST5 | S. aureus | Forward P | CAAAACACCAGTGACTGC | MLST5 |
| | | | TATGAAA (SEQ ID NO: 19) | |
| MST5 | S. aureus | Reverse P | AACATCGAGTTAATACGT | MLST5 |
| | | | GACCATTC (SEQ ID NO: 20) | |
| MST5 | S. aureus | Probe 1 | TGTTCAACGGCTGCT (SEQ | MLST5 |
| | | | ID NO: 21) | |
| MST5 | S. aureus | Probe 2 | TGTTCAACGGCTACTT | MLST5 |
| | | | (SEQ ID NO: 22) | |
| MST5 | S. aureus | Forward P | TTTAATCGCGCGTCACCAT | MLST5 |
| | | | (SEQ ID NO: 23) | |
| MST5 | S. aureus | Reverse P | AACAGCTGGCACAAATGA | MLST5 |
| | | | CAAT (SEQ ID NO: 24) | |
| MST5 | S. aureus | Probe 1 | TGTATTTAATTCAGATGCC | MLST5 |
| | | | GTT (SEQ ID NO: 25) | |
| MST5 | S. aureus | Probe 2 | ATTTAATTCAGATGCCATT | MLST5 |
| | | | G (SEQ ID NO: 26) | |
| MST5 | S. aureus | Forward P | TGTAGCTGCTTCATCATTA | MLST5 |
| | | | ATACCATT (SEQ ID NO: 27) | |
| MST5 | S. aureus | Reverse P | AACAGTTGCAGGTGTAAA | MLST5 |
| | | | TCAAGTG (SEQ ID NO: 28) | |
| 16S | S. aureus | Forward P | GGGACCCGCACAAGCGGT | 16S |
| | | | GG (SEQ ID NO: 29) | |
| 16S | S. aureus | Reverse P | GGGTTGCGCTCGTTGCGG | 16S |
| | | | GA (SEQ ID NO: 30) | |
| icaA | S. aureus | Forward P | GAGGTAAAGCCAACGCAC | icaA |
| | | | TC (SEQ ID NO: 31) | |
| icaA | S. aureus | Reverse P | CCTGTAACCGCACCAAGTT | icaA |
| | | | T (SEQ ID NO: 32) | |
| icaB | S. aureus | Forward P | ATACCGGCGACTGGGTTT | icaB |
| | | | AT (SEQ ID NO: 33) | |
| icaB | S. aureus | Reverse P | TTGCAAATCGTGGGTATGT | icaB |
| | | | GT (SEQ ID NO: 34) | |
| icaC | S. aureus | Forward P | CTTGGGTATTTGCACGCAT | icaC |
| | | | T (SEQ ID NO: 35) | |
| icaC | S. aureus | Reverse P | GCAATATCATGCCGACAC | icaC |
| | | | CT (SEQ ID NO: 36) | |
| icaD | S. aureus | Forward P | ACCCAACGCTAAAATCAT | icaD |
| | | | CG (SEQ ID NO: 37) | |
| icaD | S. aureus | Reverse P | GCGAAAATGCCCATAGTT | icaD |
| | | | TC (SEQ ID NO: 38) | |
| clfA | S. aureus | Forward P | ACCCAGGTTCAGATTCTGG | clfA |
| | | | CAGCG (SEQ ID NO: 39) | |
| clfA | S. aureus | Reverse P | TCGCTGAGTCGGAATCGCT | clfA |
| | | | TGCT (SEQ ID NO: 40) | |
| clfB | S. aureus | Forward P | AACTCCAGGGCCGCCGGT | clfB |
| | | | TG (SEQ ID NO: 41) | |
| clfB | S. aureus | Reverse P | CCTGAGTCGCTGTCTGAGC | clfB |
| | | | CTGAG (SEQ ID NO: 42) | |
| ebps | S. aureus | Forward P | GGTGCAGCTGGTGCAATG | ebps |
| | | | GGTGT (SEQ ID NO: 43) | |
| ebps | S. aureus | Reverse P | GCTGCGCCTCCAGCCAAA | ebps |
| | | | CCT (SEQ ID NO: 44) | |
| fnbA | S. aureus | Forward P | AAATTGGGAGCAGCATCA | fnbA |
| | | | GT (SEQ ID NO: 45) | |
| fnbA | S. aureus | Reverse P | GCAGCTGAATTCCCATTTT | fnbA |
| | | | C (SEQ ID NO: 46) | |
| fnbB | S. aureus | Forward P | ACGCTCAAGGCGACGGCA | fnbB |
| | | | AAG (SEQ ID NO: 47) | |
| fnbB | S. aureus | Reverse P | ACCTTCTGCATGACCTTCT | fnbB |
| | | | GCACCT (SEQ ID NO: 48) | |
| fib | S. aureus | Forward P | CGTCAACAGCAGATGCGA | fib |
| | | | GCG (SEQ ID NO: 49) | |
| fib | S. aureus | Reverse P | TGCATCAGTTTTCGCTGCT | fib |
| | | | GGTTT (SEQ ID NO: 50) | |
| eno | S. aureus | Forward P | TGCCGTAGGTGACGAAGG | eno |
| | | | TGGTT (SEQ ID NO: 51) | |
| eno | S. aureus | Reverse P | GCACCGTGTTCGCCTTCGA | eno |
| | | | ACT (SEQ ID NO: 52) | |
| cna | S. aureus | Forward P | AATAGAGGCGCCACGACC | cna |
| | | | GT (SEQ ID NO: 53) | |
| cna | S. aureus | Reverse P | GTGCCTTCCCAAACCTTTT | cna |
| | | | GAGCA (SEQ ID NO: 54) | |
| NC_002952- | S. aureus | Flanking | CATTTTTAAATTTTACAGA | 105870 |
| edit | | Sequence | CACTGTTTCCGGTATAATG | |
| | | | AAATTAATTTGAAAATTCA | |
| | | | GGAT (SEQ ID NO: 55) | |
| NC_002952- | S. aureus | Flanking | CATTTTTAAATTTTACAGA | 105870 |
| edit | | Sequence | CACTGTTTCCGCTATAATG | |
| | | | AAATTAATTTGAAAATTCA | |
| | | | GGAT (SEQ ID NO: 56) | |
| NC_002952- | S. aureus | Flanking | GAGCTTAAGGAACAAGGG | 657832 |
| edit | | Sequence | AAGATTAAAGCCGTTGGT | |
| | | | GTATCAAATTTCACATTAG | |
| | | | ATCAAC (SEQ ID NO: 57) | |
| NC_002952- | S. aureus | Flanking | GAGCTTAAGGAACAAGGG | 657832 |
| edit | | Sequence | AAGATTAAAGCCATTGGT | |
| | | | GTATCAAATTTCACATTAG | |
| | | | ATCAAC (SEQ ID NO: 58) | |
| NC_002952- | S. aureus | Flanking | AAGAATTAAGAGATAAAT | 727126 |
| edit | | Sequence | ATGAATGGAACCTCAGTTT | |
| | | | ACTCTCTAAAATTGACAAC | |
| | | | TACCT (SEQ ID NO: 59) | |
| NC_002952- | S. aureus | Flanking | AAGAATTAAGAGATAAAT | 727126 |
| edit | | Sequence | ATGAATGGAACCCCAGTT | |
| | | | TACTCTCTAAAATTGACAA | |
| | | | CTACCT (SEQ ID NO: 60) | |
| NC_002952- | S. aureus | Flanking | TCAAAGATGTAACTACATT | 908100 |
| edit | | Sequence | TTATGAGGAAGGTAAACA | |
| | | | TTTAATCTATGGTTATACA | |
| | | | CCAAC (SEQ ID NO: 61) | |
| NC_002952- | S. aureus | Flanking | TCAAAGATGTAACTACATT | 908100 |
| edit | | Sequence | TTATGAGGAAGATAAACA | |
| | | | TTTAATCTATGGTTATACA | |
| | | | CCAAC (SEQ ID NO: 62) | |
| NC_002952- | S. aureus | Flanking | TTTCTGCATGTCGAGGATT | 1186436 |
| edit | | Sequence | TTTAACATAACTGTTTGTG | |
| | | | TCAGTTAGTTTTAACTTTT | |
| | | | TACT (SEQ ID NO: 63) | |
| NC_002952- | S. aureus | Flanking | TTTCTGCATGTCGAGGATT | 1186436 |
| edit | | Sequence | TTTAACATAACGGTTTGTG | |
| | | | TCAGTTAGTTTTAACTTTT | |
| | | | TACT (SEQ ID NO: 64) | |
| NC_002952- | S. aureus | Flanking | CAACCTAGACGTGAATGG | 1419668 |
| edit | | Sequence | ATTGAAAAGCATTTTGAGT | |
| | | | TTGGTATGCAAGAGGACC | |
| | | | AAAGTA (SEQ ID NO: 65) | |
| NC_002952- | S. aureus | Flanking | CAACCTAGACGTGAATGG | 1419668 |
| edit | | Sequence | ATTGAAAAGCATGTTGAG | |
| | | | TTTGGTATGCAAGAGGAC | |
| | | | CAAAGTA (SEQ ID NO: 66) | |
| NC_002952- | S. aureus | Flanking | TCTTCCCCGACCCAGTCAT | 1656006 |
| edit | | Sequence | CAGCATCATCAGGGCTTA | |
| | | | CACCATTCGCTTCACCAAC | |
| | | | AGTCA (SEQ ID NO: 67) | |
| NC_002952- | S. aureus | Flanking | TCTTCCCCGACCCAGTCAT | 1656006 |
| edit | | Sequence | CAGCATCATCAGGCTTAC | |
| | | | ACCATTCGCTTCACCAACA | |
| | | | GTCA (SEQ ID NO: 68) | |
| NC_002952- | S. aureus | Flanking | ATAGTCACAGCTTCTTTAA | 1762538 |
| edit | | Sequence | AATGAGTTATGACTTCATC | |
| | | | AATATTCTCATTTTTCATA | |
| | | | AATA (SEQ ID NO: 69) | |
| NC_002952- | S. aureus | Flanking | ATAGTCACAGCTTCTTTAA | 1762538 |
| edit | | Sequence | AATGAGTTATGGCTTCATC | |
| | | | AATATTCTCATTTTTCATA | |
| | | | AATA (SEQ ID NO: 70) | |
| NC_002952- | S. aureus | Flanking | TTATCTGTTTCTGCTTGCG | 1897263 |
| edit | | Sequence | CTTCTTTCTTCACTTCTTGA | |
| | | | ATCGCTTGTGCTTCTTGTG | |
| | | | ATG (SEQ ID NO: 71) | |
| NC_002952- | S. aureus | Flanking | TTATCTGTTTCTGCTTGCG | 1897263 |
| edit | | Sequence | CTTCTTTCTTCGCTTCTTGA | |
| | | | ATCGCTTGTGCTTCTTGTG | |
| | | | ATG (SEQ ID NO: 72) | |
| NC_002952- | S. aureus | Flanking | CAAATCCAAAACAATTTG | 2209374 |
| edit | | Sequence | ACGTCATCGTTTACGAAA | |
| | | | ACTTATTTGGCGATATTTT | |
| | | | AAGTGA (SEQ ID NO: 73) | |
| NC_002952- | S. aureus | Flanking | CAAATCCAAAACAATTTG | 2209374 |
| edit | | Sequence | ACGTCATCGTTTGCGAAA | |
| | | | ACTTATTTGGCGATATTTT | |
| | | | AAGTGA (SEQ ID NO: 74) | |
| NC_002952- | S. aureus | Flanking | GCAAGAACACATTTAGTA | 2211044\hat{ } |
| edit | | Sequence | TCCCCTGCTATGGCAGCAG | 2211045 |
| | | | CAGCTATTCATGGTAAATT | |
| | | | TGTG (SEQ ID NO: 75) | |
| NC_002952- | S. aureus | Flanking | GCAAGAACACATTTAGTA | 2211044\hat{ } |
| edit | | Sequence | TCCCCTGCTATGGCAGCAG | 2211045 |
| | | | CAGCAGCTATTCATGGTA | |
| | | | AATTTGTG (SEQ ID NO: 76) | |
| NC_002952- | S. aureus | Flanking | GATATTAGCATTCATACGT | 2257298 |
| edit | | Sequence | TTGAACTCTGCGACATGCA | |
| | | | TAAAACGATTTTCAAATAC | |
| | | | AGTT (SEQ ID NO: 77) | |
| NC_002952- | S. aureus | Flanking | GATATTAGCATTCATACGT | 2257298 |
| edit | | Sequence | TTGAACTCTGCAACATGCA | |
| | | | TAAAACGATTTTCAAATAC | |
| | | | AGTT (SEQ ID NO: 78) | |

### Exemplary Embodiments

In one embodiment, the allelic discrimination panel detects Staphylococcus, a species of Staphylococcus, a strain of Staphylococcus, or a combination thereof. In one embodiment, the panel detects a highly pathogenic Staphylococcus, e.g., one that is hypertransmissible. In one embodiment, the panel amplifies specific nucleic acid in a sample from a mammal, e.g., a human. In one embodiment, the panel detects MLST 5, MLST 8, MLST 15, MLST 30, or MLST 59. In one embodiment, the panel detects a polymorphism at one or more of the following positions in S. aureus: 189723, 716410, 1517201, 1517381, 64695, 64954, 64974, 65001, 65096, 65216, 66001, 66200, 66543, 105870, 657832, 727126, 908100, 1186436, 1419668, 1656006, 1762538, 1897263, 2209374, 2211044, or 2257298. In one embodiment, the panel employs a probe comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a polynucleotide with at least 90%, 95%, 98% or 99% nucleic acid sequence identity thereto, or a combination thereof.

In one embodiment, the panel a method of identifying and/or monitoring a pathogenic bacterial strain among commonly isolated intraoperative multilocus sequence types is provided. The method includes obtaining a sample from a mammal, e.g., a human such as a patient in a hospital or clinic and performing an MLST assay on the sample or a portion thereof. The sample may be an oral sample, a fecal sample, a blood sample or any tissue sample from the mammal. The assay includes, in one embodiment, amplifying nucleic acid that is specific for Staphylococcus, e.g., specific for MLST 5, MLST 8, MLST 15, MLST 30, or MLST 59. The assay includes, in one embodiment, amplifying nucleic acid that is specific for Staphylococcus, and probing the amplified nucleic acid with a probe that is specific for MLST 5, MLST 8, MLST 15, MLST 30, or MLST 59. In one embodiment, the probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a combination thereof. In one embodiment, the assay detects a polymorphism at position one or more of the following positions in S. aureus: 189723, 716410, 1517201, 1517381, 64695, 64954, 64974, 65001, 65096, 65216, 66001, 66200, 66543, 105870, 657832, 727126, 908100, 1186436, 1419668, 1656006, 1762538, 1897263, 2209374, 2211044, or 2257298.

Also provided is a kit having one or more of the primers or probes disclosed herein.

Further provided is an array for amplification and detection of nucleic acid from a pathogenic bacterial strain, wherein in at least one address on the array has a probe or at least one primer specific for MLST5. In one embodiment, the address has dried probe and at least one dried primer. In one embodiment, the address has an aqueous solution with the probe and at least one primer. In one embodiment, the probe has at least 80%, 82%, 85%, 87%, 90%, 92%, 95%, 98% or 99% identity to one of SEQ ID Nos. 1-8, 13-14, 17-18, 21-22, or 25-26. In one embodiment, at least one primer has at least 80%, 82%, 85%, 87%, 90%, 92%, 95%, 98% or 99% identity to one of SEQ ID Nos. 15-16, 19-20, 23-24 or 27-28. In one embodiment, the probe is specific for a polymorphism at position 189723, 716410, 1517201, 1517381, 64695, 64954, 64974, 65001, 65096, 65216, 66001, 66200, 66543, 105870, 657832, 727126, 908100, 1186436, 1419668, 1656006, 1762538, 1897263, 2209374, 2211044, or 2257298 in the bacterial genome.

Further provided is a method of using the array, including contacting the address with a physiological sample from a human under conditions that allow for the at least one primer to hybridize to a template and produce a copy of the template, for amplification of the copy and the template amplification to occur and for the probe to hybridize to the amplified nucleic acid if the sample has the pathogenic bacteria; and detecting whether the probe hybridized to the address in the array. In one embodiment, the sample is a blood sample, a skin sample, a fecal sample, an oral sample, a biopsy, or a bronchial sample.

In one embodiment, a method of detecting a pathogenic bacterial strain in a sample is provided. The method includes hybridizing a probe comprising has at least 80% identity to one of SEQ ID Nos. 1-8, 13-14, 17-18, 21-22, or 25-26 to nucleic acid in a sample; and detecting whether the probe hybridizes to the sample. In one embodiment, the sample is amplified nucleic acid. In one embodiment, at least one primer for the amplification has at least 80% identity to one of SEQ ID Nos. 15-16, 19-20, 23-24 or 27-28. In one embodiment, the sample is a physiological sample from a human. In one embodiment, the sample is a sputum sample or is from a wound of the human. In one embodiment, if the probe hybridizes to the sample, the human is infected with the pathogenic bacteria and in one embodiment, is treated for that infection.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

### Determining Probe Sequences and Phenotypic Background for an Allelic Discrimination Panel for S. aureus

An allelic discriminating panel was created for S. aureus by using 178 S. aureus isolates that were collected from 274 randomly selected operating room environments. Both the phenotype and genotype of the isolates were analyzed.

Phenotypic analysis included biotype identification, biofilm formation, and multidrug resistance. Biotype was identify using the combination of colony morphology, Gram stain, and simple rapid test using a BIOMERIEUX API® system

Biofilm analysis was performed by growing the isolates overnight in tryptic soy broth, at about 37° C., shaking at about 200 rpm, and with maximum aeration. The same day the strains start their growth, each well of a 48-well incubation plate was coated with about 200 μl of about 20% human plasma and stored at about 4° C. The following day and after incubation, each strain was inoculated at about 1:1000 into brain-heart infusion medium with about 0.4% glucose, and each well of the 48-well plate was aspirated. Then about 800 μl of each strain in the glucose treated media was added to each well and incubated at 37° C. overnight. The following day, the culture fluid was aspirated from each well, each well was washed twice with about 800 μl of phosphate-buffered saline, and then fixed with 70% ethanol. Plates were then air dried for about 10 minutes and then stained with about 800 μl of about 0.1% crystal violet for about five minutes. The crystal violate was aspirated and the wells washed again with PBS. The stain was eluted in about 800 μl of isopropanol for about 30 minutes and about 200 μl was transferred to a microtiter plate. Absorbance of each well was measured at 595-630 nm. Each isolate was analyzed four time with the average absorbance at 620 nm normalized using a sample average of SarA.

To obtain the drug resistances of each isolate, disk diffusion antibiotic susceptibility testing was used with methicillin, ampicillin, cefazolin, cefepime, ceftazidime, cefuroxime, meropenem, piperacillin-tazobactam, penicillin, ciprofloxacin, clindamycin, and vancomycin. Methicillin and vancomycin resistance was confirmed by agar dilutional minimal inhibitory concentration. Isolates with resistance to all but vancomycin were defined as multi-drug resistant (MDR) isolates.

Desiccation tolerance was measured by using two to three colonies from each of 173 pure S. aureus cultures were used to inoculate 100 mL of Brain-heart infusion (BHI) broth and incubated overnight (18-24 hours) at 35° C. One mL of BHI culture was then placed into a 1.5 mL centrifuge tube and spun at 13,000 rpm to generate a pellet. The supernatant was discarded, and the pellet was washed two times with 500 μL phosphate buffered saline (PBS). Bacteria were resuspended in PBS to OD600=0.1 (in standard spectrometer with light path of about one cm) and vortexed. To determine the CFU/mL for day zero, 20 μL of each bacterial dilution were added to 180 μL of PBS for each of three wells of a 96-well cell culture plate for each isolate. This serial dilution process was extended to 108. Ten μL of 105 through 108 dilutions were then used to inoculate each of three blood agar plates in triplicate on each plate for each sample well (1 isolate placed into 3 wells and plated in triplicate on each plate=three plates for each isolate with nine rows of sample dilutions in total) and incubated overnight (18-24 hours) at 35° C. Twenty μL of each bacterial suspension were also placed on the inner side of a wide open lid of each of six 1.5 mL Eppendorf tubes for each sample (three tubes for the first day after preparation and three tubes for the second day after preparation). All tubes were allowed to dry completely and were subsequently placed into an empty drawer sheltered from dust and air to allow desiccation to occur. On days one and two, one mL of PBS was added to each of the tubes, the tubes closed, inverted, the dried droplet allowed to dissolve in the PBS for 15 minutes, and each tube vigorously vortexed four times for five seconds. The serial dilutions for 101-103 were repeated in triplicate (3 wells) for each isolate, the undiluted sample through 103 dilutions transferred to blood agar plates, and the plates incubated overnight at 35° C. Colony counts were obtained from the dilution that provided two to 20 discrete colonies, the results were averaged across the three wells for each isolate, and the colony forming unit (CFU)/mL was determined by the following equation: CFU/mL=Average number of colonies for a dilution×100×dilution factor. The top 25% of day two CFU/mL results defined greater desiccation tolerance. Results were also recorded as the day 2 proportion of inoculum survival.

To compare the genomes of each isolate, the DNA from each of the 178 was extracted sequenced using next generation sequencing. The sequences were then assembled, and the assemblies were then compared. DNA was extracted and then diluted to 1.2 μg/60 μl. The diluted DNA was then sheared using ultrasonication into about 400 bp fragments. Libraries were then made by performing end repair, A-tailing, ligation, and amplification. Each library was prepared using an adapter that carried a unique barcode. Equimolar amounts of each library were pooled and analyzed for fragment size, and fragments from 450-670 bp were recovered. Clusters were then generated and the flow cell was loaded onto the ILLUMINA® system for sequence analysis.

Following sequencing, the sequences were trimmed by removing the adapters, and then used to identify S. aureus 252 (MRSA252, NC 002952) as the best reference sequence match. Other libraries may be used to select their own reference sequence match. Consensus sequences were then generated from the aligned sequences, and then the consensus sequences were used to generate multilocus sequence types (MLSTs). MLSTs were then based on the sequence of seven genes. This example produced 22 known and 12 novel MLSTs, for a total of 34 unique MLSTs.

The MLSTs were then statistically associated to the results of the phenotypic analysis above to determine high risk MLSTs. Of the 34 unique MLSTs, MLST 5, and 8 were found to be the most pathogenic, having strong associating with biofilm formation and MDR. S. aureus isolates were stratified by day 2 CFU/mL, and clonal complexes for the top 25% were assessed to characterize MLST involvement by site and frequency.

Sequence types (ST) identified for >2 occurrences within clonal complexes (ST 5, 15, 30, and 105) were examined for an association with biofilm formation and desiccation tolerance according to two-sided Wilcoxon-Mann-Whitney tests. For biofilm formation, P-values were Bonferroni corrected for three comparisons. The relative expression of 12 genes involved in biofilm formation (Table 3) were compared between ST 5 and all other isolates in the upper quartile of biofilm formation using two-sided, two-groups Student T-tests with unequal variances (i.e., Welch's t-tests). The P-values were Bonferroni corrected for the 12 multiple comparisons. The association of ST with biofilm formation with multidrug resistance, chi-square tests were used to test the association between multidrug resistance and each of the above listed demographic covariates, except age that was test using t-test. Logistic regression was then used to examine the relationship between multidrug resistance and the independent variable of ST 5 while adjusting for covariates with P≤0.05 in the chi-square analysis. Fisher's exact test was used to compare the proportion of infection cultures that were ST 5 as compared to all other MLST.

To determine the expression of the biofilm associated genes, the RNA was quantified using qPCR. Cultures were grown overnight in TSB at 37° C. to an OD₆₀₀ of 1 to 1.5. One milliliter of each sample was centrifuged at 8000 rpm for 2 min with removal of the supernatant. Pellets were resuspended with 92 μL of 100 mM Tris buffer (pH 8.0) and 8 μL of lysostaphin (10 mg/mL in 20 mM sodium acetate buffer, pH 4.5). Resuspended cells were incubated at room temperature for 30 minutes, inverted 3 times during incubation, and 1 μL Proteinase K (Qiagen, Hilden, Germany) added followed by an additional 30 minutes for cell lysis. A Qiagen RNeasy Mini Kit (Qiagen, Hilden, Germany) was used according to protocol for RNA isolation and purification. The eluted and purified RNA was quantified on a NanoDrop 2000 Spectrophotometer (Thermo Scientific).

The High Capacity cDNA Reverse Transcription Kit with RNase Inhibitor (Applied Biosystems, Vilnius, Lithuania) was used according to protocol with a maximum of 200 ng/μL of RNA utilized. Duplicate reverse transcription reactions were run for each sample in a 96-well plate and the products transferred to 0.5 mL microcentrifuge tubes and stored at −20° C.

All cDNA samples were diluted to 0.95 ng/μL in IL buffer, pH 8.0, and 10 μM working dilutions of the 12 biofilm gene and 16S primers were made in IL buffer, pH 8.0. A1:1 mix of the forward and reverse working primer solutions was prepared for each set, with each sample and control run in triplicate 10 μL reactions in 384-well plates. Each reaction contained 5 μL Fast SYBR Green master mix (Applied Biosystems by Thermo Fisher Scientific, Vilnius, Lithuania), 0.4 μL 10 μM forward and reverse primer mix, 2.5 μL molecular-grade water, and 2.1 cDNA or molecular-grade water in the no template controls. The 384-well plates were sealed with an optical adhesive cover and centrifuged 1 min at 1,200 RPM before being placed in the Quant Studio 6 Flex Real-Time PCR System (Applied Biosystems by Life Technologies, Singapore, and Singapore) for thermal cycling. The plates were run using the Quant Studio Real-Time PCR Software v1.1's comparative CT setting with melt curve analysis.

For desiccation tolerance, P-values were adjusted using the Bonferroni correction for multiple comparisons for the 4 ST. Adjustment for potentially confounding variables was planned. The preceding covariates were assessed using the Wilcoxon-Mann-Whitney or Spearman rank correlation [age], with the dependent variable being the day 2 CFU/ml measurements. One covariate was potentially significant (P≤0.05), cardiothoracic/vascular procedures. Logistic regression analysis was then used to determine whether ST 5 remained associated with greater desiccation tolerance as defined as the top quartile of day 2 CFU/mL measurements, while adjusting for the covariate.

The association of transmitted ST 5 isolates with the mecA resistance trait and methicillin resistance was examined using logistic regression analysis while adjusting for covariate(s) with P≤0.05 in the chi-square or t-test (age) analyses, specifically site 0, site 2, general abdominal surgery, plastics/breast surgery, and inpatient origin.

Calculations were performed using Stata. All P-values and confidence intervals (CI) were 2-sided. No additional power analyses were conducted for this study, as all 173 S. aureus isolates were included in the analysis.

For biofilm formation, 22, S. aureus ST types were isolated from intraoperative reservoirs, ST 5, 8, 15, 30, and 59 accounted for approximately 71% (127/178) of isolates. ST 5 (IRR_{adj} 6.67, 95% CI 1.82-24.41, P=0.0008), 8 (IRR_{adj} 8.33, 95% CI 2.31-30.12, P=0.0001), and 15 (IRR_{adj} 5.73, 95% CI 1.35-24.33, P=0.0009) were associated with increased risk of clonally-associated transmission while adjusting for potentially confounding variables.

ST 5 was associated with greater biofilm absorbance [(ST 5 median absorbance 3.08, SD 0.642) vs. (other ST median absorbance 2.38, SD 1.01), Mann-Whitney Test corrected P=0.021]. The expression of 12 genes known to be associated with biofilm formation was compared for ST 5 vs. all other isolates in the top quartile of biofilm absorbance. The expression of fnbB was increased for ST 5 [(MLST 5 ΔΔCT 24.76, SD 0.53) vs. Other ST ΔΔCT 20.83, SD 4.82), corrected P=0.001].

For desiccation tolerance, the top quartile of desiccation tolerant isolates was comprised of five clonal complexes and 7 distinct isolates that involved a total of 12 ST. ST 5, 15, 30, and 105 accounted for 67% (31/46) of desiccation tolerant ST (Table 3).

S. aureus ST type 5 isolates had greater desiccation tolerance (day 2 CFU/mL) as compared to all other ST [(MLST 5, N=34, median day 2 CFU survival 0.027%±0.029%), (other MLST, N=139, median day 2 CFU survival 0.0091%±1.41%), corrected P=0.0001]. ST 5 isolates that were transmitted had greater desiccation tolerance as compared to all other ST [(ST 5, N=15, median day 2 CFU survival 0.023%±0.037%), (other ST, N=158, median day 2 CFU survival 0.0099%±0.132%), P=0.022). ST 5 remained associated with the upper quartile of desiccation tolerance after adjusting for cardiothoracic/vascular procedures (OR 4.22, 95% CI 1.91-9.31, P=0.0004).

ST 5 was associated with multidrug resistance while adjusting for origin of isolate (OR 7.82, 95% CI 2.19-27.95, P=0.002). Six of 12 patient infection cultures were ST 5. ST 5 was associated with increased risk of patient infection as compared to all other ST 96/38 MLST 5 vs. 6/140, RR 3.68, 95% CI 1.26-10.78, P=0.022).

### Example 2

### Allelic Discrimination Panels for S. aureus

A real-time polymerase chain reaction (PCR) card was made for the common intraoperative S. aureus MLSTs discovered above. The panel comprises a microfluidic card, reagents, probes, primers, sample, and plate seal. The microfluidic card is a 384-well card with the MSLT probe and primers freeze dried in each well. Two microfluidic cards were designed, the first to assay for the common intraoperative S. aureus MLST, including those for MRSA and MSSA, and the second to assay MLST5. In addition, each assay on the card would have positive and negative controls run in triplicate (6 controls each assay). The 6 control assays are run in triplicate (18) for each sample. Three patient samples are run in triplicate for a total of 84 sample being run preop.

The fill ports of the card are then loaded with a master mix, comprising 20 mM Tris-HCl (pH 8.4) and 50 mM KCl at a 1× concentration. The dNTPs were at a final concentration of 0.2 mM each. The magnesium source had a final concentration of 1.5 mM. The polymerase used was a Taq polymerase. The passive reference dye was ROX™.

The sample was a crude bacterial lysate obtained from a swab. Different methods were used to collect the samples. For some samples, a dry swab was inserted about 1 inch into each patient nares and rolled 4 times. For other samples, the swab was rolled 4 times within the patient axilla; a provider's, attending physicians, physician assistants, nurses, or orderlies, dominate palmer surface; and environmental surfaces, adjustable pressure-limiting value and agent dial of the anesthesia machine. Once the initial swab is made, the swab is inserted into a glass transport tube containing 1 mL Aimes Transport Medium. The tube was then vortexed for 15 seconds, and then 850 μL was transferred to a separate lysis tube. The lysis tube was then centrifuged at 21,000×g for 5 minutes, the resultant supernatant removed, and 50 μL of lysis buffer, made up of 20 mM Tris.Cl, pH 8.0, 2 mM sodium EDTA, and 1.2% Triton® X-100, was added to the pellet. The cells were lysed at this point and the resulting sample was added to the master mix. The master mix was then aliquoted into the fill ports of the cards, and then the cards were centrifuged to move the

master mix, including the sample, into the corresponding wells, containing the pre-filled primers and probes. After centrifugation, the card was then loaded onto a PCR machine capable of reading the reporter attached to the probe. Real-time PCR was then run according to the following protocol: a pre-read stage at 60.0° C. for 30 second for 1 cycle; and initial hold stage at 95.0° C. for 20 seconds for 1 cycle; the PCR stage with the denaturing stage at 95.0° C. for 1 second and a joint annealing and extension step with data collection at 60.0° C. for 20 seconds for 40 cycles; and a final post-read stage at 60.0° C. for 30 seconds for 1 cycle. All primers and probes were designed to anneal at 60.0° C.

The results of the PCR run were then analyzed to determine the alleles present in the sample. This determination was then used to determine the MLST of the S. aurous strain present within the sample.

### Example 3

### Boosting Bacterial Counts Prior to Lysis

In another embodiment of the panel, the swab of the above example was inserted into 1 mL of growth media, comprising of tryptic soy broth with 6.5% NaCl and incubated for 6 hours at 35° C. prior to being put into the lysis protocol.

### Alternate Methods of Bacterial Lysis

In another embodiment of the panel, one or more lysis techniques were combined. This included, but is not limited to, using lysostaphin and detergent treatment, freeze/thaw cycle, sonication, microbead disruption, and boiling.

### Example 4

### Allelic Discrimination

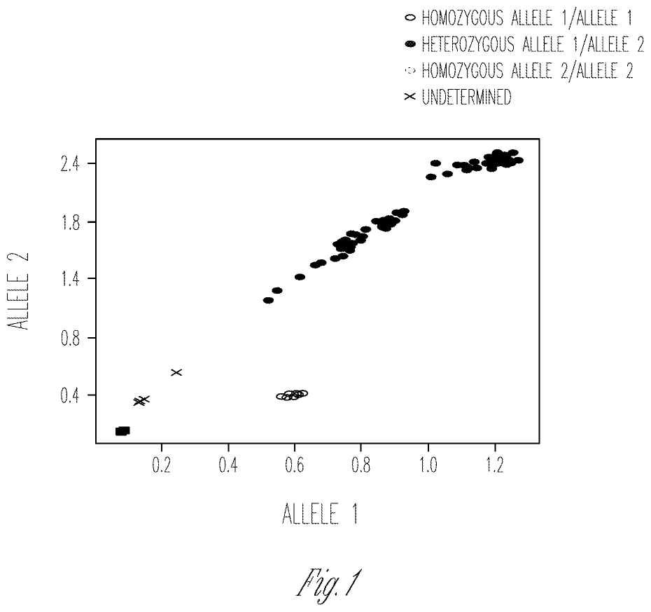

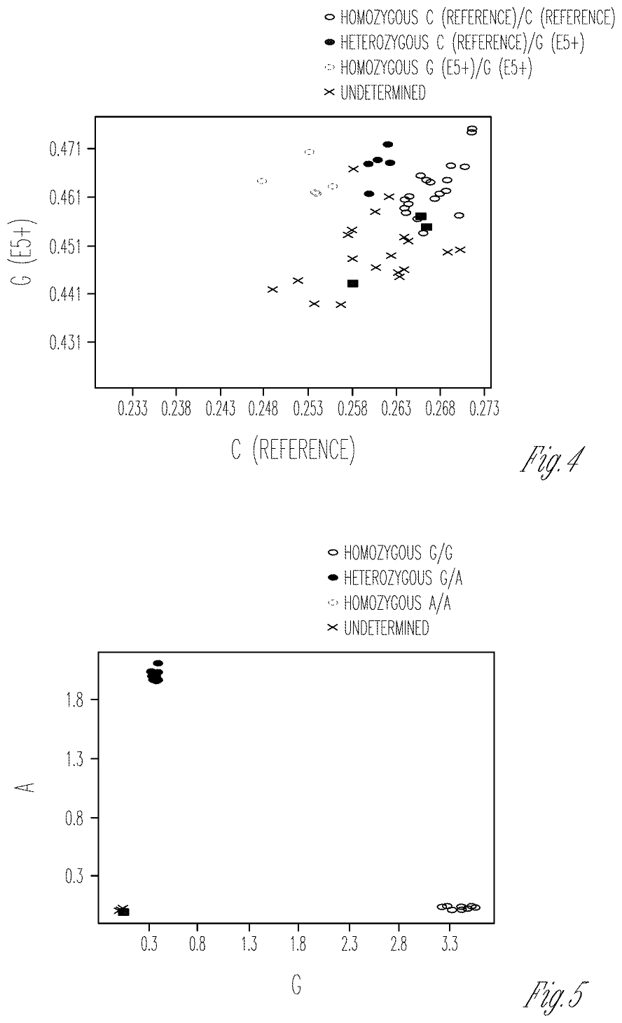

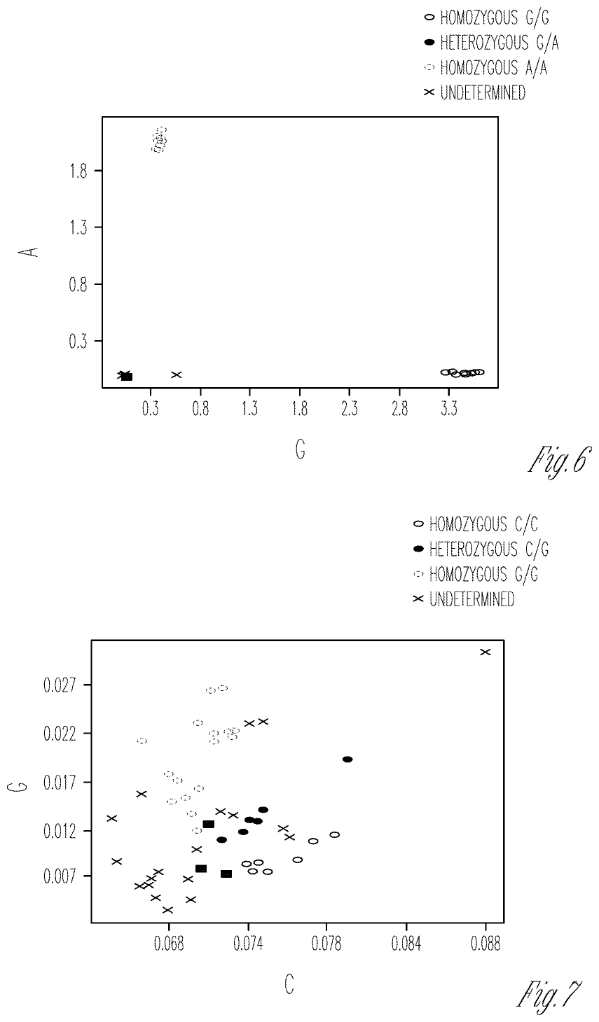

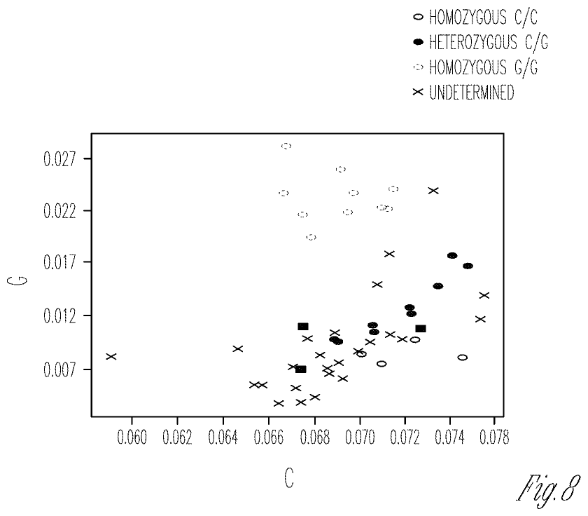

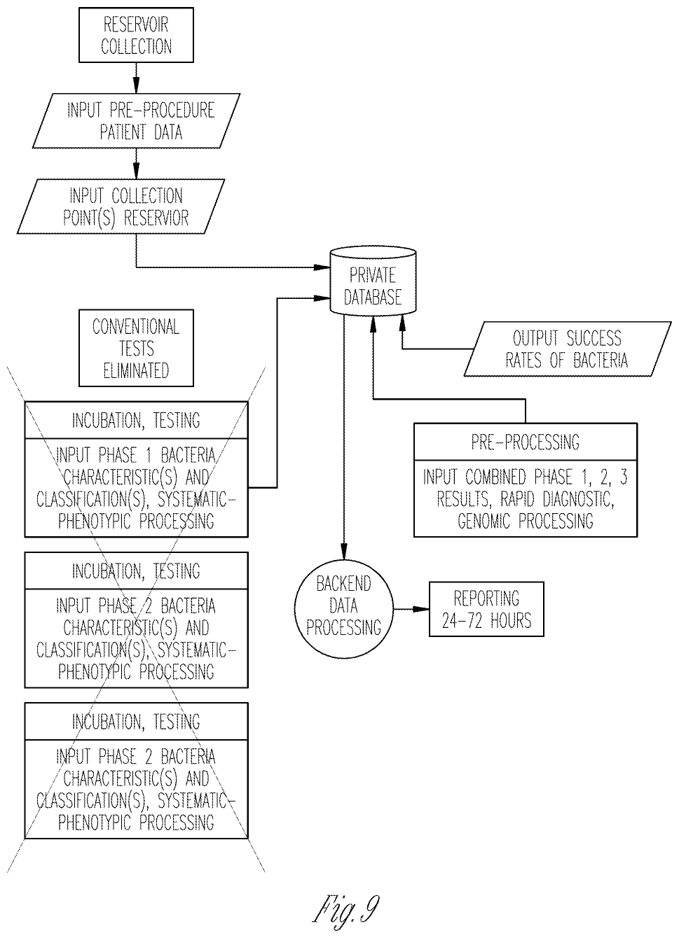

One embodiment of the methods and allelic discrimination assay was performed to assess the allelic discrimination panel. Multiple alleles were tested for discrimination and the results are provided in FIGS. 1-8.

To determine the discriminatory ability of the probes, the wells of a PCR card were preloaded with PCR probes and primers. Sample of either isolates, non-staph isolates (bacterial negative controls), or water (negative control) and master mix were then loaded onto the cards and PCR was carried out. As shown in the figures, the panels were able to produce tight clusters of isolates with the same genotypes. Undetermined were either the negative controls or the bacterial negative controls.

### Example 5

S. aureus pathogens have evolved to acquire genetic traits that are associated with increased antibiotic resistance, virulence, and transmissibility (Boucher et al., 2009). As a result, there has been an alarming increase in the spread of these invasive pathogens from acute care settings to healthy members of the community (Klevens et al., 2007).

The operating room patient care arena is contributing to this problem. Perioperative S. aureus nasal carriage occurs frequently (von Eiff et al., 2001; Loftus et al., 2015), is associated with intraoperative transmission in up to 39% of cases (Loftus et al., 2015), and has been directly linked to postoperative bacteremia in patients undergoing orthopedic surgery (von Eiff et al., 2001).

There are evidence-based solutions that can address this alarming patient safety issue. Preoperative S. aureus decolonization has been shown to reduce the incidence of surgical site infections (Bode et al.; Schweizer et al., 2015). Novel hand hygiene improvement measures (Koff et al., 2009), disinfectable needleless closed catheter devices (Loftus et al., 2012a), and catheter hub disinfection prior to injection have been shown to reduce intraoperative transmission and subsequent postoperative infection development (Loftus et al., 2012b). However, despite this solid foundation of evidence, intraoperative adherence to these evidence-based, basic preventive measures is abysmal (Koff et al., 2009; Loftus et al., 2012a; Loftus et al., 2012b; Loftus et al., 2012c).

As a result of suboptimal compliance with these basic preventative measures, at least 7% of patients undergoing surgery will acquire 1 or more healthcare-associated infections (HAIs) (Vogel et al., 2012; Koff et al., 2016), with S. aureus is a leading cause (Klevens et al., 2007; von Eiff et al., 2001; Loftus et al., 2015; Bode et al.; Schweizer et al., 2015). The Centers for Disease Control, World Health Organization, and the White House consider HAis to be a devastating and persistent issue linked to antibiotic resistance, and they have urged healthcare providers to tighten compliance with basic preventive measures in order to prevent infections and unnecessary antibiotic use.13-15

### Materials and Methods:

### Background/General Description:

A computer-generated list was used to randomly select 274 case-pairs (first and second case of the day in each of 274 operating room environments) from three academic medical centers in the United States. The randomized unit study design was intended to include a wide variety of surgical procedures, patient comorbidities, infection control measures, and health care providers. More than 8,184 bacterial pathogens were collected over a one year period in order to capture seasonal variation (Loftus et al., 2015; Loftus et al., 2012c). As the activity was limited to analysis of de-identified data from a previous IR.B approved project (201507774, Assessment of Routine Intraoperative Horizontal Transmission of Potentially Pathogenic Bacterial Organisms II), the University of Iowa waived the need for IR.B review.

A total of 178 S. aureus isolates were collected. One hundred seventy-three isolates were implicated in possible transmission, defined as at least two S. aureus isolates identified from two distinct reservoirs within or between cases in an intraoperative case-pair. An additional 5 isolates were identified in postoperative patient infection cultures without a possible intraoperative link (Loftus et al., 2015; Loftus et al., 2012c).

Institutional infection control policies were tracked and recorded during the study period. At all centers, usual infection control practices included routine and terminal environmental cleaning involving quaternary ammonium compounds±surface disinfection wipes. All providers had access to alcohol dispensers located on the wall and/or anesthesia carts, and gloves were immediately available for use. There were no changes in these usual procedures during the study period (Loftus et al., 2015; Loftus et al., 2012c).

S. aureus Reservoir Collection Process and Analysis of Transmission:

The study unit was a case-pair which involved the first and second case of the day in a given operating room environment. Bacterial reservoirs were systematically sampled over time in order to employ the platform of temporal association (Loftus et al., 2015; Loftus et al., 2012c; Loftus et al., 2008; Loftus et al., 2018). Temporal association was defined as organisms collected from two or more distinct reservoirs in the same operating room on the same day at the same time (Loftus et al., 2015; Loftus et al., 2012c; Loftus et al., 2008; Loftus et al., 2018).

The conceptual framework for the utilization of temporal association in the model was considered as follows: 1) Prior to phenotypic or genomic testing to examine relatedness, two isolates obtained from two or more distinct reservoirs within a study unit were considered more likely to be related than independent given that the probability of S. aureus isolation from any one tested site ranged from 3 (hand and environmental samples) to 16 (patient nasopharynx and/or axilla) percent (Loftus et al., 2012c). 2) Thus, the probability of isolating S. aureus from two distinct reservoirs within the platform of temporal association, probability of Ax B, was considered to range from 0.09 to 3%, while the probability of being related to a common reservoir was considered to range from 97-99.91%. 3) The reservoirs sampled included anesthesia provider [attending and resident physicians and Certified-Registered Nurse Anesthetists (CRNA)] hands before, during, and after patient care, environmental sites proven to reliably represent the magnitude of contamination of the anesthesia work environment (Loftus et al., 2008), patient skin sites strongly correlated with surgical site infections (Loftus et al., 2008), and the internal lumen of open lumen intravascular stopcock sets (Loftus et al., 2015; Loftus et al., 2012c; Loftus et al., 2008; Loftus et al., 2018). Air was considered a continuous medium in the model that could impact all reservoirs in parallel through settling of aerosolized particles. 4) Temporal association of reservoirs in a given study unit was applied according to the following sequence of events: Environmental sites (adjustable pressure-limiting valve and agent dial of the anesthesia machine) were decontaminated and subsequently cultured to establish a baseline (time 0). Anesthesia provider hands were then sampled upon operating room entry (time 1). After induction of anesthesia and patient stabilization, the patient nasopharynx and axilla were sampled (time 3). Provider hands (any provider that entered the anesthesia workspace outside of the sterile field including but not limited to anesthesia providers and technologists) were sampled during care (time 4). The same environmental sites were sampled at case end (time 5) along with the internal lumen of the patient intravenous stopcock set (time 6), and provider hands were again sampled at case end (time 7). This process was repeated for the second case in an observational unit, except that environmental sites were not decontaminated in order that residual contamination following routine cleaning procedures between-cases could be assessed. The specific methods of culture acquisition and handling for this process are previously described (Loftus et al., 2015; Loftus et al., 2012c; Loftus et al., 2008; Loftus et al., 2018).

Temporal association was then strengthened by a systematic phenotypic and genomic approach. First, temporally-associated isolates from at least two distinct reservoirs in a study unit underwent phenotypic testing and molecular typing to identify epidemiologically-related isolates (Steps 1 and 2). Simple rapid tests, analytical profile indexing, antibiotic susceptibility testing, and multi.locus sequence typing were conducted as previously described (Loftus et al., 2018). Epidemiologically-related isolates then underwent whole cell genome analysis and single nucleotide polymorphism analysis (Step 3).

Clonally-related isolates were aligned according to the timing of culture acquisition (Step 4). Provider origin of within-case transmission was confirmed if the transmitted isolate was clonally-related to an isolate from the hands of 1 or more anesthesia providers sampled upon room entry before patient care. Provider origin of between-case transmission was confirmed if one or more isolates from provider hands in case 1 were clonally-related to one or more isolates in case 2 without potential alternative sources of transmission from case 2 reservoirs. Environmental origin of within-case contamination was confirmed if the transmitted isolate was clonally-related to an isolate from the environment sampled at baseline or at case end but not isolated either from the hands of providers or from the patient at case start. Environmental origin of between-case transmission was confirmed if one or more environmental isolates from case 1 were clonally-related to one or more isolates in case 2 without potential alternative sources of transmission from case 2 reservoirs. Patient origin of within-case contamination was confirmed if the transmitted isolate was clonally-related to an isolate from the patient sampled at case start but was not isolated either from the hands of providers at case start (as patient samples were obtained after induction of anesthesia) or from baseline environmental samples. Patient origin of between-case transmission was confirmed if one or more patient isolates from case 1 were clonally-related to one or more isolates in case 2 without potential alternative sources of transmission from case 2 reservoirs. The within-case mode of transmission was confirmed if the origin and transmission location(s) for a clonal series were confined to a single case in a study unit. The between-case mode of transmission was confirmed if the clonal series spanned both cases in a study unit (Loftus et al., 2015; Loftus et al., 2012c; Loftus et al., 2008; Loftus et al., 2018).

Isolates involved in one or more transmission stories were coded as being involved in a transmission (step 5).

### Assessment of Biofilm Formation:

S. aureus isolates (N=178) were grown overnight in tryptic soy broth (Soybean-Casein Digest Medium, Becton, Dickinson and Company, New Jersey 07417) at 37° C., shaking at 200 revolutions per minute (rpm) with maximum aeration. On day 1, each well in a forty-eight well plate (Costar 48-Well Culture Cluster, Flat Bottom with Lid) was coated with 200 μL of 20% human plasma (Sigma-Aldrich, St. Louis, Mo. 63178) and stored at 4° C. On day 2, strains were inoculated 1:1,000 into brain-heart infusion (BHI) medium (Research Products International Corp., Mount Prospect, Ill., 60056) with 0.4% glucose (Research Products International Corp., Mount Prospect, IL 60056). For each well of the 48-well plate, the plasma was aspirated, mixed with 800 μL of a unique strain, added back to the well, and incubated at 37° C. overnight. On day 3, the culture fluid was aspirated from each well and each well washed 2 times with 800 μL phosphate-buffered saline (DPBS, Gibco by Life Technologies, Grand Island, N.Y. 14072) and fixed with 800 μL 70% ethanol. Plates were allowed to dry for IO minutes and then stained with 800 μL of 0.1% crystal violet (Fisher Chemical, Fair Lawn, N.J. 07410) for 5 minutes. The crystal violet was aspirated and the wells washed again with PBS. The stain was eluted in 800 μL of isopropanol for 30 minutes and 200 μL transferred to a new microtiter plate. Absorbance of each well was then measured at 595-630 mn using a Tecan infinite M200 plate reader. This experiment was repeated 4 times for each isolate with isolate-specific results summarized as the average absorbance reading for each of the 4 measurements at 620 mn and normalized by the sample average for a known low biofilm performer control (SarA) (Archer et al., 2011; Hekiert et al., 2009).

The expression of 12 genes known to be associated with biofilm formation” was quantified for isolates in the top quartile of biofilm absorbance. The relative expression of the 12 genes was then compared for ST 5 vs. other ST. The methodology for the analysis of expression of the 12 genes can be found in the appendix.

Multidrug Resistance:

Antibiotic susceptibility was performed on all isolates. Multidrug resistance was defined as resistance to methicillin, cefazolin, ampicillin, cefepime, ceftazidime, cefuroxime, ciprofloxacin, clindamycin, meropenem, penicillin, and piperacillin-tazobactam.

Postoperative Infections:

All patients were for followed for 30 days in the postoperative period to for infection surveillance (Loftus et al., 2015; Loftus et al., 2012c; Loftus et al., 2008; Loftus et al., 2018). Initial screening included elevated white blood cell count, fever, antiinfective order, culture, or office documentation of signs of infection. If ≥ criteria were present, the patient underwent a full chart review by the principal investigator at each hospital site to determine whether patients met criteria for a healthcare-associated infection as defined by the National Healthcare Safety Network (Edwards et al., 2009). All cultures obtained for infection workup were saved for comparison to intraoperative reservoir isolates.

### Demographic Data:

Basic patient, procedural, and provider demographic information collected including the hospital [labeled 0, 1, or 2], age, sex, American Society of Anesthesiologists (ASA) physical status >2, patients with >2 comorbidities, type of surgery (cardiothoracic/vascular, orthopedic, gynecology/oncology, general abdominal, plastics/breast, and other), dirty or infected site, duration of greater than 2 hours, Study on the Efficacy of Nosocomial Infection control (SENIC) (Haley et al., 1980) score (an index predicting the probability of postoperative HAI development for a given patient)>2, urgent surgery, and patient origin and discharge locations including the hospital floor, intensive care unit, or postanesthesia care unit.

### Statistical Analysis:

Pathogenic strains were defined by hyper transmissibility, increased strength of biofilm formation, increased risk of multidrug resistance, and increased risk of infection. Identification of pathogenic S. aureus ST was systematically addressed as follows.

To identify hyper transmissible ST, chi squared tests were used to examine the potential association of each of the covariates mentioned above with clonally-related transmission, except that age was tested using t-test. Poisson regression with robust variance was then used to estimate the incidence risk ratios (IRR) of any transmission event for the independent variables of frequently encountered intraoperative MLST including MLST 5, MLST 8, MLST 15, MLST 30, and MLST 59, while adjusting for covmiate(s) with P:S 0.05 in the chi-square analysis, specifically general abdominal surgery. Poisson regression was used to estimate the risk ratio because the incidence of transmission (33.5% of N=173 isolates potentially linked to intraoperative transmission) was so large that the odds ratio estimated using logistic regression would be a biased estimator of the relative risk (Prasad et al., 2008; Zou, 2004; Lindquist, 2018). These 5 two-sided P-values and confidence intervals were adjusted using Bonferroni correction for the 5 multi-locus sequence groups.

Wilcoxon-Mann-Whitney tests were used to compare the strength of biofilm formation for hyper transmissible ST, specifically ST 5, 8, and 15. These two-sided P-values were Bonferroni connected for the three comparisons. The relative expression of 12 genes involved in biofilm formation were compared between ST-5 and all other isolates in the upper quartile of biofilm formation using two-sided, two-group Student t-tests with unequal variances (i.e., Welch's t-tests). The P-values were Bonferroni corrected for the 12 multiple comparisons.

In order to test the association of hyper transmissible ST with greater strength of biofilm formation (ST 5) with multidrug resistance, chi-square tests were used to test the association between multidrug resistance and each of the above listed demographic covariates, except age that was tested using t-test. Logistic regression was then used to examine the relationship between multidrug resistance and the independent variable of ST 5, while adjusting for covariates with P s 0.05 in the chi-square analysis including site 0, site 2, general/abdominal, and plastic/breast surgery. Fisher's exact test was used to compare the proportion of infection cultures that were ST 5 as compared to all other MLST. Finally, transmission dynamics for S. aureus ST 5 were assessed and characterized by OR PathTrac (RDB Bioinformatics, Omaha, Nebr. 68154).

Calculations were performed using Stata 15.1 (StataCorp LLC, College Station, Tex.). No additional power analyses were conducted for this study, as all available S. aureus isolates were included in the analysis.

### Results

Twenty-two S. aureus ST types were isolated from intraoperative reservoirs. ST 5, 8, 15, 30, and 59 accounted for approximately 71% (127/178) of isolates. ST 5 (IRRadj 6.67, 95% CI 1.82-24.41, P=0.0008), 8 (IRR adj 8.33, 95% CI 2.31-30.12, P=0.0001), and 15 (IRR adj 5.73, 95% CI 1.35-24.33, P=0.009) were associated with increased risk of clonally-associated transmission while adjusting for potentially confounding variables (Table 4).

ST 5 was associated with greater biofilm absorbance [(ST 5 median absorbance 3.08, SD 0.642) vs. (other ST median absorbance 2.38, SD 1.01), Mann-Whitney Test corrected P=0.021]. The expression of 12 genes known to be associated with biofilm formation was compared for ST 5 vs. all other isolates in the top quartile of biofilm absorbance. The expression of fnbB was increased for ST 5 [(MLST 5 ΔΔCT 24.76, SD 0.53) vs. Other ST ΔΔCT 20.83, SD 4.82), corrected P 0.001].

ST 5 was associated with multidrug resistance while adjusting for hospital sites 0 and 2, general surgery, and plastic/breast surgery (OR 7.82, 95% CI 2.19-27.95, P=0.002) (Table 5). Six of 12 patient infection cultures were ST 5 (Table 6). ST 5 was associated with increased risk of patient infection as compared to all other ST (6/38 MLST 5 vs. 6/140, RR 3.68, 95% CI 1.26-10.78, P=0.022).

Overall, 6 of 12 of the postoperative S. aureus infections were linked to organisms cultured from intraoperative reservoirs at the time of the surgery, with anesthesia provider hands and patient skin surfaces implicated as intraoperative sources by whole cell genome analysis.

Overall transmission dynamics for the more pathogenic ST 5 were assessed, including affected operating room environments and failure mode analysis indicating need for improvement in basic measures. Seven transmission stories were identified. Patients (N=4) and provider hands (N=3) were confirmed as clonal sources of intraoperative ST 5 transmission. Provider hands (N=3), patients (N=4), and environmental surfaces (N=2) were identified as clonal transmission locations. All observed transmission stories involved the within-case mode of transmission. Two ST 5 transmission stories were linked to postoperative infection development by whole cell genome analysis.

TABLE 4
Frequently Encountered Intraoperative S. aureus
MLST and Risk of Clonally-Related Transmission

| | IRR | Corrected 95% CI | Corrected P-value |
|---|---|---|---|
| MLST 5 | 6.67 | 2.48-17.89 | 0.0008 |
| MLST 8 | 8.33 | 3.14-22.15 | 0.0001 |
| MLST15 | 5.73 | 1.91-17.22 | 0.0094 |
| MLST30 | 3.04 | 1.01-9.15 | 0.26 |
| MLST59 | 4.43 | 1.37-14.33 | 0.066 |
| General abdominal | 0.61 | 0.30-1.22 | 0.83 |

MLST = multilocus sequence type,
IRR = incidence rate ratio,
CI = confidence interval

TABLE 5
The Association of S. aureus ST 5 with Multidrug-Resistance

| | OR | 95% | P-value |
|---|---|---|---|
| MLST 5 | 7.82 | 2.19-27.95 | 0.002 |
| Site 0 | 11.30 | 1.20-106.05 | 0.034 |
| Site 2 | 1.51 | 0.13-16.92 | 0.33 |
| Plastic/Breast Surgery | 2.81 | 0.82-9.61 | 0.10 |

ST == sequence type,
OR == odds ratio,
CI == con:fidence interval.

Among the 39 S. aureus isolates that were obtained from patients undergoing general abdominal surgery, 0% had multidrug resistance. This perfect prediction of lack of resistance included 8 of the 34 ST 5 isolates linked to intraoperative transmission, Table 2 shows the results of the remaining 134 cases including 26 ST 5 isolates.

TABLE 6
Systematic Phenotypic and Genomic Surveillance
of Postoperative 5. aureus Infections

| | | | |
|---|---|---|---|
| | | Genomic | |
| Sequence Type | Culture Type | Confirmation | Source |

Postoperative S. aureus Infection Cultures
Linked by Systematic Phenotypic and Whole
Cell Genome Analysis to Intraoperative
Reservoir Cultures Prior to Surgery

| | | | |
|---|---|---|---|
| 5 | Sputum | Yes | Patient |
| 5 | Unknown | Yes | Patient |
| 8 | Wound | Yes | Resident Hand |
| 8 | Wound | Yes | Patient |
| 8 | Wound | Yes | Patient |
| 30 | Respiratory | No | Unknown |
| 188 | Unknown | Yes | Patient |

Postoperative S. aureus Infection Cultures
without Intraoperative Source

| | | | |
|---|---|---|---|
| 5 | Wound | No | Unknown |
| 105 | Wound | No | Unknown |
| 5 | Unknown | No | Unknown |
| 5 | Sputum | No | Unknown |
| 5 | Swab | No | Unknown |

### DISCUSSION

Attenuation of S. aureus transmission is an important target for postoperative infection prevention (Boucher et al., 2009; Klevens et al., 2007; von Eiff et al., 2001; Loftus et al., 2015; Bode et al.; Schweizer et al., 2015; Loftus et al., 2012c; Loftus et al., 2018). This study has identified S. aureus ST 5 is a pathogenic strain associated with increased risk of transmission, greater strength of biofilm formation, increased antibiotic resistance, and increased risk of postoperative infection development. This study of transmission dynamics for this key strain identified patient skin surfaces and provider hands as sources of intraoperative ST 5 transmission. Thus, improved preoperative patient decolonization and perioperative hand hygiene infection control measures may help to control the spread of this important strain characteristic. In addition, continual assessment of the quality of routine and terminal environmental cleaning protocols is indicated because residual contamination of operating room environmental surfaces was implicated in ST 5 clonal transmission.

S. aureus ST 5 is associated with USAIO0, a cause of invasive, hospital acquired infections that occur frequently, especially in patients with risk factors for HAI development (Klevens et al., 2007; Schultea et al., 2013). We have shown that intraoperative ST 5 isolates are hyper transmissible, antibiotic resistant, and virulent. These results explain at least in part why USA100 is considered a more pathogenic strain (Schultea et al., 2013). The hyper transmissibility of ST 5, combined with an advancing population age and more complex surgical procedures (Dellinger and Gordon, 2006), could help to explain the increasing community spread of invasive MRSA infections when patients acquire such pathogens during routine, intraoperative care. Thus, the impact of intraoperative infection control efforts targeting ST 5 attenuation on the incidence of invasive MRSA infections should be assessed.

ST 5 transmissibility, antibiotic resistance, and virulence may be related to increased strength of biofilm formation. This work shows that this involves increased production of fibronectin binding protein B through expression of fnbB (McCourt et al., 2014). Fibronectin binding protein is known to impact the attachment and accumulation phases of biofilm formation, key properties that could augment biofilm formation on both environmental and implanted device surfaces (McCourt et al., 2014). This is an important finding, as in the setting of improper environmental cleaning, this strain characteristic could facilitate intraoperative transmission and postoperative infection development. Thus, more aggressive monitoring of the efficacy of routine and terminal operating room cleaning activities is indicated. Routine surveillance of operating room transmission via OR Path Trac can identify operating rooms exposed to more pathogenic strains, such as ST 5, that are in need of improved quality and frequency of cleaning (Loftus et al., 2018). These efforts could be extended to monitor the efficacy of cleaning of equipment such as laryngoscope handles that are often insufficient (Caffau and Nadali, 1965; Friss and Helms, 1963; Maslyk et al., 2002; Madar et al., 2005; Foweraker, 1995; Leung and Chan, 2006; Edmiston et al., 2005; Fukada et al., 1996) to reduce the number of colony forming units in the patient care environment that could lead to infection (Caffau and Nadali, 1965; Friss and Helms, 1963; Maslyk et al., 2002; Madar et al., 2005; Foweraker, 1995; Leung and Chan, 2006; Edmiston et al., 2005; Fukada et al., 1996).

Multiple studies have shown that improved intraoperative hand hygiene and vascular care practices are desperately needed in the operating room to prevent vascular contamination with pathogens that are primarily derived from provider hands (Loftus et al., 2015; Koff et al., 2009; Loftus et al., 2012a; Loftus et al., 2012b; Loftus et al., 2012c; Loftus et al., 2018). In a randomized, ex vivo study, anesthesia providers were estimated to inject up to 50,000 colony forming units (CFU) of bacterial pathogens in a series of 5 sterile injections, where Staphylococcus was the most frequently injected pathogen.” Considering ST 5 hyper transmissibility, provider hands as a key source of transmission, and therefore the heightened risk of intravascular injection, this work confirms the need for a high degree of compliance with basic preventive measures such as proper hub disinfection and syringe handling akin to monitoring of compliance with SCIP measures (Stulberg et al., 2010).

ST 5 hyper transmissibility is made worse by the association with multidrug resistance. While ST 5 has been previously tied to methicillin resistance (Carrel et al., 2015; Noto et al., 2008), we show that ST 5 is associated with pan resistance to antibiotics commonly administered prior to the surgical incision, such as cefazolin and cefuroxime. Such resistance is an important distinguishing factor, as a patient colonized with this sequence type could undergo the following series of events: 1) the primary skin incision compromising their first line of defense, 2) ST 5 entry directly to the wound from aerosolized particles, directly from intraoperative vectors, or through injection through intravascular devices with seeding of the wound hematoma, 3) immunosuppression resulting from both the anesthetic agents employed and the surgical inflammation (Procopio et al., 2001), 4) provision of an ineffective antibiotic, 5) bacterial attachment to implanted hardware, devices, hematomas, and/or necrotic tissue, 6) increased bacterial expression of fnbB facilitating attachment and accumulation (McCourt et al., 2014) resulting in stronger biofilm formation, 7) a quiescent state that shields the pathogen from antibiotics, even if effective, 8) a Th1 inflammatory response further compromising host defenses induced by the biofilm itself (Hekiert et al., 2009), and 9) the development of an acute and/or chronic infection (McCourt et al., 2014). Thus, it is important to understand the epidemiology of pathogens that are resistant to commonly employed antibiotic agents in order that basic practices involved in the transmission of those pathogens can be optimized, continually, and so proper antibiotics can be administered. This could be achieved by ongoing surveillance of transmission of S. aureus as previously described (Loftus et al., 2018).

Given the prevalence of ST 5 across multiple institutions as shown in this study, combined with the well described noncompliance with basic preventive measures in the operating room likely contributing to aerosolization of particles and settling (Caffau and Nadali, 1965; Friss and Helms, 1963; Maslyk et al., 2002; Madar et al., 2005; Foweraker, 1995; Leung and Chan, 2006; Edmiston et al., 2005; Fukada et al., 1996; Reddy and Loftus, 2018), it is not surprising that we were able to confirm with whole cell genome analysis that provider hands and patient skin surfaces are key sources for the intraoperative spread of ST 5 isolates. Based on these results, improved patient decolonization and provider hand hygiene practices should be included in initial intraoperative infection control strategies for the ST 5 strain group. While we have provided exciting results that show genotyping is an important consideration for improved patient decolonization, a first step is to extend evidence-based decolonization involving nasal mupirocin (Bode et al.; Schweizer et al., 2015), chlorhexidine (Climo et al., 2013), and/or povidone iodine (Phillips et al., 2014) from orthopedic and cardiothoracic patients to a larger patient population. This is especially important because prior work has clearly demonstrated that bacteria colonizing one patient can affect another patient undergoing care in the same arena (Loftus et al., 2015; Loftus et al., 2012c). These efforts could be augmented in parallel with evidence-based hand hygiene and catheter care techniques (Koff et al., 2009; Loftus et al., 2012a; Loftus et al., 2012b). While monitoring of operating room traffic could be helpful (Young and O'Regan, 2010), monitoring would likely need to be tied to a meaningful surveillance endpoint to be effective in attenuating the multiple contributors to total operating room air CFU counts (Parikh et al., 2010; Shaw et al., 2018). One approach would be to link surveillance of the multiple reservoirs known to impact air quality to meaningful surveillance outcomes, such as operating room contamination with ST 5, in order that meaningful feedback could be provided along with plan-do-study-act cycles to reduce traffic (Loftus et al., 2018).

In conclusion, S. aureus ST 5 is a hyper transmissible, strong biofilm forming, antibiotic resistant, and virulent genotype that is frequently encountered in today's operating room environments. Patient skin surfaces and provider hands are confirmed sources and OR environmental surfaces confirmed transmission locations. Improved patient decolonization, intraoperative hand hygiene, and environmental cleaning may help to control the spread of this important pathogen in order to improve intraoperative patient safety.

### Example 6

Surgical site infections are a subset of healthcare-associated infections (HAis) that affect 3-5% of all patients undergoing surgery and are associated with a 2-fold increase in patient morbidity and modality, a 2-fold increase in hospital duration, and a 66% increase in the risk of intensive care unit (ICU) admission (Vogel et al., 2012; Koff et al., 2016; Kirkland et al., 1999; Bode et al., 2010)—don't include Kirk). The Centers for Disease Control (CDC) and the World Health Organization (WHO) consider HAis as a devastating issue tied to antibiotic resistance and have highlighted three major goals for HAI prevention including the following: 1) Prevention of infections in patients undergoing surgery, 2) Prevention of patient-to-patient bacterial transmission, and 3) improvement in antibiotic stewardship (Boucher et al., 2009; WHO, 2014; MMWR, 2001).

These recommendations apply to the perioperative arena where the contribution of intraoperative bacterial reservoirs to bacterial transmission events and postoperative infection development has been confirmed (Loftus et al., 2015; Burgess et al., 2016). Indeed, a multicenter study recently demonstrated that stopcock contamination occurred in 23% of surgical cases, was associated with increased modality, and was linked by molecular typing to postoperative infection (Loftus et al., 2015). Intraoperative bacterial reservoir isolates have also been directly linked to the causative organism of infection for 30% of 30-day postoperative HAIs. Finally, transmission of S. aureus has been confirmed in up to 39% of surgical cases and provider hands, environmental surfaces, contaminated stopcock sets, and patient skin sites have been directly linked to postoperative infection development (Potts, 1994). Attenuation of perioperative S. aureus transmission is an important target for HAI reduction.

Individually, improvements in patient decolonization, provider hand hygiene, intravascular catheter design and handling, and environmental cleaning have reduced infection and improved intraoperative patient safety. Prior work in the operating room determined that anesthesia provider hands were an important vector for high-risk stopcock transmission events (Burgess et al., 2016). At Dartmouth, this finding led to hand hygiene improvements that reduced stopcock contamination and postoperative HAIs (Wielders et al., 2002; Loftus et al, 2012c). It has also been shown that improved catheter design can significantly reduce bacterial injection as compared to conventional open lumen devices (Loftus et al., 2008), and that a novel disinfection approach can reduce stopcock contamination and postoperative infections and phlebitis (Loftus et al., 2018). Intraoperative environmental contamination (Hasman et al., 2014) patterns have been mapped and have led to the development of an improved intraoperative environmental cleaning procedure (Zerbino et al., 2008).

Recent evidence has evaluated the relationship between bacterial strain characteristics and risk of clonally-related, intraoperative transmission using a new information technology (IT) program, OR PathTrac (RDB Bioinformatics, Omaha, Nebr. 68154). This program integrates bacterial isolate temporal association achieved via systematic reservoir collection with simple rapid tests, analytical profile indexing, antibiotic sensitivity profiles, multilocus sequence typing, and single nucleotide variant analysis to yield clonal alignment and typical transmission patterns for hyper transmissible, vimlent, and resistant pathogens. Bacterial source(s) that require greater attention in a particular context are identified and a feedback loop provided to generate continual, proactive optimization of infection control measures. This tool leverages advanced technology to address uncertainly in relative reservoir contribution that as suggested by Colbeck regarding methicillin-susceptible Staphylococcus aureus (MSSA), would otherwise require reliance on the dangerous post hoc ergo proper hoc argument.

A recent study that used OR PathTrac to examine intraoperative MRSA transmission found that patients frequently served as a reservoir of origin for within and between-case transmission events, and the hands of attending anesthesiologists and residual contamination of anesthesia machines following routine cleaning were important vectors for between-case transmission events. This provided evidence-based improvement pathways to address key CDC agenda. In addition, MRSA isolates were shown to be hyper transmissible as compared to MSSA isolates, indicating methicillin resistance as an important strain characteristic in need of focused attention in the operating room.

S. aureus desiccation tolerance is one such strain characteristic. In order to cause infection, a S. aureus isolate must survive long enough to spread from its reservoir to its host. Several factors can impact S. aureus survival and affect the size of the inoculum, including desiccation tolerance. Prior observations have shown that 10⁸-10⁹ CFU of desiccation tolerant MRSA isolates can last up to 180-360 days in the environment. It has also been shown that enhanced environmental survival of these isolates on provider hands, plastic, and metal surfaces can lead to increased rates of transmission. Conceptually, transmission of more desiccation tolerant isolates could subsequently spread antibiotic resistance traits and increase the risk of postoperative infection development.

This is an important consideration in the operating room where increased survival following infection control measures could leave an inoculum on provider hands, patient skin surfaces, and/or environmental sites that could have an immediate impact on risk of transmission and infection development in the OR where there are up to 350 patient-provider-environmental interactions per hour. As an example, aerosolization (talk about equipment, case 1 to case 2) As such, it is important to investigate the potential relationship between desiccation tolerance, bacterial spread, and infection to advance intraoperative infection control efforts.

### Materials and Methods

### Background/General Description

Two hundred seventy-four operating room environments were randomly selected for observation at 3 United States academic medical centers [9]. The observational unit was a case pair including the first and second case of the day in each operating room so within and between-case S. aureus transmission could be detected. As study activity was limited to analysis of de-identified data from the previous IRB approved project (201507774, Assessment of Routine Intraoperative Horizontal Transmission of Potentially Pathogenic Bacterial Organisms II), the University of Iowa declared that the additional analysis in the current study did not meet the definition of human subjects research.

Infection control practices included routine and terminal environmental cleaning with quaternary ammonium compounds±surface disinfection wipes. All providers had access to alcohol dispensers located on the wall and/or anesthesia carts, and gloves were immediately available for use. There were no changes in these usual procedures during the study period.

General Overview of S. aureus Reservoir Collection Process Among Study Units

S. aureus isolates (N=173) were recovered from operating room reservoirs including environmental sites at baseline and at case end, healthcare provider hands throughout care, and patients after induction of anesthesia and stabilization. Patients were followed for 30 days to assess for HAI development, and S. aureus isolates identified as causative organisms of infection were compared to intraoperative isolates obtained during the time of surgery.

### Isolate Comparison

S. aureus isolates present at case end that were not present at case start were considered transmitted. Temporal association, analytical profile indexing, antibiotic susceptibility testing, multilocus sequence typing, and single nucleotide variant analysis were used to compare>2 isolates obtained from distinct reservoirs within an observational unit. OR Path'Irac (RDB Bioinformatics, Omaha, Nebr. 68154) was used to integrate these results to identify clonally-related isolates for the purpose of generating transmission maps. Pulsed-field gel electrophoresis (PFGE) was used as an additional typing method to compare intraoperative isolates to causative organisms of infection.

Clonally-related isolates were defined as epidemiologically-related isolates (temporal association with analytical profile indexing and antibiotic susceptibility testing match) with >99.99% agreement in. single nucleotide variant (SNV) analysis. Infection links also required an indistinguishable PFGE banding pattern. Greater than 99.99% agreement in single nucleotide variants corresponded to 85±54 SNV for clonally-related isolates while isolates of the same MLST had 1270±340.SNV differences.

### Sample Collection Technique:

Hand Sampling.

A previously validated, modified glove juice technique was utilized to sample provider hands before, during, and after patient care (Loftus et al., 2012c; Loftus et al., 2018).

Patient Sampling.

The patient's nasopharynx was sampled to assess the patient reservoir because nasopharyngeal pathogens have been strongly associated with postoperative surgical-site infections (Hasman et al., 2014). The patient's axilla was also sampled because the axilla harbors up to 15%-30% of pathogens colonizing patient skin (Zergino and Birney, 2008).

Environmental Sampling.

The adjustable pressure-limiting valve and agent dial of the anesthesia machine were sampled. These sites have been previously associated with an increase in the probability of bacterial contamination of patient intravenous stopcock sets (Wielders et al., 2002). These sites were sampled at baseline after active decontamination at case start for case 1 and after routine decontamination at case start for case 2. They were sampled again at the end of the case 1 and case 2 via Dimension III (Butcher's, Sturtevant, Wis.) disinfectant solution according to manufacturer's recommendations. Active decontamination involved targeted cleaning of the study sites by the study investigators using a quaternary ammonium compound strictly according to the manufacturer's protocol allowing 10 minutes for air drying which was not mandated for routine cleaning.

### Microbial Culture Conditions:

All culturing was done in the same laboratory at Site 0. Samples shipped from Sites 1 and 2 were placed under similar environmental conditions (ambient temperature) during the 12 hours required for shipping. Samples collected on the same day at Site 0 did not require shipping but were kept at ambient temperature to mimic the environment of those samples being shipped. No samples for a given study day were incubated until all samples for that day from all research sites were present at Site 0.

### Systematic-Phenotypic-Genomic Analysis:

### Temporal Association:

Two S. aureus isolates obtained from two or more distinct reservoirs within a study unit were considered temporally associated because they were more likely to be related than independent given that the probability of S. aureus isolation from any one tested site ranged from (hand and environmental samples) to 16 (patient nasopharynx and/or axilla) percent. Thus, the probability of isolating S. aureus from two distinct reservoirs within the platform of temporal association, probability of Ax B, was considered to range from 0.09 to 3%, while the probability of being related to a common reservoir was considered to range from 97-99.91%.

### Analytical Profile Indexing:

Bacterial organisms were identified and isotypes specified using the commercially available bioMerieux API identification system (Marcy l'Etoile, France), resulting in a 7-9 digit identification number. This number was then cross referenced using the Analytical Profile Index database to obtain the final organism biotype (Burgess et al., 2016; Potts, 1994; Loftus et al., 2018).

### Antibiotic Susceptibility:

Disk diffusion antibiotic susceptibility testing analysis was employed. Bacterial sensitivity was recorded and subsequently analyzed as sensitive or resistant (including intermediate resistance) (Loftus et al., 2018). Methicillin resistant Staphylococcus aureus (MRSA) and vancomycin-resistant enterococcus (VRE) were also confirmed by agar dilution minimal inhibitory concentration (Burgess et al., 2016; Potts, 1994; Loftus et al., 2018).

S. aureus isolates that were temporally associated and of the same biotype and antibiotic susceptibility profiles underwent genomic testing as described below.

### Next-Generation Sequencing:

DNA was extracted, and next generation sequencing was performed at the Iowa Institute of Human Genetics (IIHG) using the Illumina platform. DNA samples (1.2 ug/60 ul) were sheared 240 to about 400 bp fragments on the Covaris E220. Sequencing libraries were prepared from the sheared DNA (1 ug/50 ul) using the KAPA Hyper Library Prep on the PE Caliper Sci clone (Rosche Diagnostics, Indianapolis, Ind. 46250-0457). Each library was prepared using an adapter that carries a unique barcode (Integrated DNA Technologies, Coralville, Iowa 52241). Libraries were analyzed on a fragment analyzer and equimolar amounts of the libraries were pooled based on fragment analyzer results for a smear analysis of 450-670 bp. A size range of 450-670 bp was recovered from the pool on the Blue Pippin (Sage Science, Beverly, Mass. 01915). The KAPA library quantification kit for Illumina platforms was used to determine the molar concentration of the size-selected pool. The pool was loaded on the cBot (Illumina) for cluster generation and the flow cell loaded on the HiSeq4000 (Illumina) for sequence analysis.

### MLST:

S. aureus sequences reads were generated and downloaded into the CLC Genomics Workbench Module (Version 1.1, Qiagen Aarhus, Getman town, MD 20874). CLC Genomics Workbench Plugin (CLC Genomics Module Version 1.1, Qiagen Aarhus, Germantown, Md. 20874) was used to trim and to remove adapters and broken pairs from S. aureus. sequence reads, and K-mer spectra analysis was utilized to identify a best match to S. aureus isolates. S. aureus 252 (MRSA252, NC_002952) was identified as the best reference sequence match. All trimmed S. aureus sequence reads were subsequently mapped to the MRSA252 complete genome. Consensus sequences for each read map were analyzed by multilocus sequence typing analysis (CLC Genomics Module Version 1.1, Qiagen Aarhus, Germantown, Md. 20874) (Hasman et al., 2014; Zerbino et al., 2008).

### Whole Cell Genome Analysis:

S. aureus read maps underwent resequencing analysis to identify insertions, deletions, and structural variants which were analyzed by fixed ploidy variant detection (CLC Genomics Workbench Plugin (CLC Genomics Module Version 1.1, Qiagen Aarhus) to identify sequence-specific nucleotide polymorphisms (SNP) (Hasman et al., 2014; Zerbino et al., 2008). Greater than 99.99% agreement in isolate single nucleotide variants was required for clonal-relatedness, a threshold supported by PFGE typing and the range of variants in unrelated isolates of the same MLST type (Zerbino et al., 2008).

### Desiccation Tolerance:

Two to three colonies from each of 173 pure S. aureus cultures were used to inoculate 100 mL of Brain-heart infusion (BHI) broth and incubated overnight (18-24 hours) at 35° C. One mL of BHI culture was then placed into a 1.5 mL centrifuge tube and spun at 13,000 rpm to generate a pellet. The supernatant was discarded, and the pellet was washed two times with 500 μL phosphate buffered saline (PBS). Bacteria were resuspended in PBS to OD₆₀₀=0.1 (in 275 standard spectrometer with light path of about one cm) and vortexed. To determine the CFU/mL for day zero, 20 μL of each bacterial dilution were added to 180 μL of PBS for each of three wells of a 96-well cell culture plate for each isolate. This serial dilution process was extended to 10⁸. Ten μL of 10⁵ through 10⁸ dilutions were then used to inoculate each of three blood agar plates in triplicate on each plate for each sample well (1 isolate→3 wells→plated in triplicate on each plate=three plates for each isolate with nine rows of sample dilutions in total) and incubated overnight (18-24 hours) at 35° C. Twenty μL of each bacterial suspension were also placed on the inner side of a wide open lid of each of six 1.5 mL Eppendorf tubes for each sample (three tubes for the first day after preparation and three tubes for the second day after preparation). All tubes were allowed to dry completely and were subsequently placed into an empty drawer sheltered from dust and air to allow desiccation to occur. On days one and two, one mL of PBS was added to each of the tubes, the tubes closed, inverted, the dried droplet allowed to dissolve in the PBS for 15 minutes, and each tube vigorously vortexed four times for five seconds. The serial dilutions for 10¹-10³ were repeated in triplicate (3 wells) for each isolate, the undiluted sample through 10³ dilutions transferred to blood agar plates, and the plates incubated overnight at 35° C. Colony counts were obtained from the dilution that provided two to 20 discrete colonies, the results were averaged across the three wells for each isolate, and the colony forming unit (CFU)/mL was determined by the following equation: CFU/mL=Average number of colonies for a dilution×JOO×dilution factor. The top 25% of day two CFU/mL results defined greater desiccation tolerance. Results were also recorded as the day 2 proportion of inoculum survival.

### Transmission Dynamics:

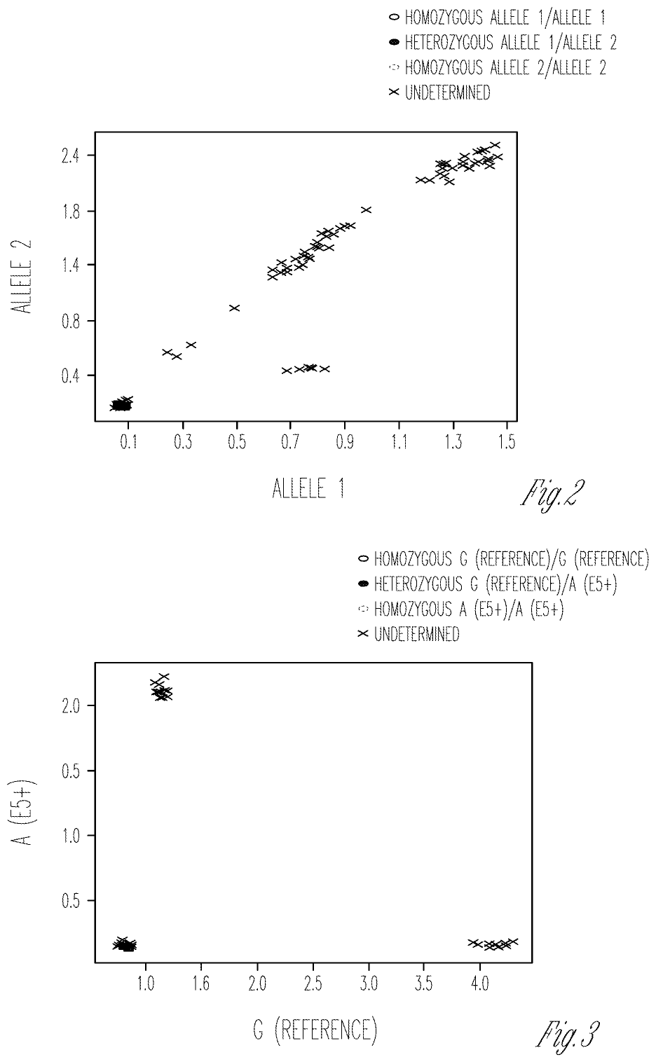

OR PathTrac integrated systematic phenotypic and genomic results to identify clonally-related isolates for each case-pair, and related isolates were aligned according to 14 distinct collection time points (FIG. 1). Case pairs were processed in aggregate to report typical transmission patterns stratified by strain characteristics, A typical transmission pattern for more desiccation tolerant isolates is shown (FIG. 2).

### Postoperative Infections:

All patients were for followed for 30 days in the postoperative period to for infection surveillance. Initial screening included elevated white blood cell count, fever, anti-infective order, culture, or office documentation of signs of infection. If one criteria were present, the patient underwent a full chart review by the principal investigator at each hospital site to determine whether patients met criteria for a healthcare-associated infection as defined by the National Healthcare Safety Network. All cultures obtained for infection workup were saved for comparison to intraoperative reservoir isolates as described above.

### Demographic Data:

Basic patient, procedural, and provider demographic information collected including the hospital [labeled 0, 1, or 2], age, gender, American Society of Anesthesiologists (ASA) physical status >2, patients with >2. comorbidities, type of surgery (cardiothoracic/vascular, orthopedic, gynecology/oncology, general abdominal, plastics/breast, and other), dirty or infected site, duration of greater than 2 hours, Study on the Efficacy of Nosocomial Infection control (SENIC) score (an index predicting the probability of postoperative HAI development for a given patient) >2, urgent surgery; and patient origin and discharge locations including the hospital floor, intensive care unit, or postanesthesia care unit.

### Statistical Analysis:

S. aureus isolates were stratified by day 2 CFU/mL, and clonal complexes for the top 25% were assessed to characterize MLST involvement by site and frequency.

ST identified for >2 occurrences within clonal complexes (ST 5, 15, 30, and 105) were examined for an association with desiccation tolerance according to two-sided Wilcoxon-Mann-Whitney tests. P-values were adjusted using the Bonferroni correction for multiple comparisons for the 4 ST. ST that remained associated with desiccation after correction (ST 5) underwent further analysis of transmitted isolates (clonally-related) of that type to determine association with desiccation using a two-sided Wilcoxon-Mann-Whitney test. Adjustment for potentially confounding variables was planned. The preceding covariates were assessed using the Wilcoxon-Mann-Whitney or Spearman rank correlation [age], with the dependent variable being the day 2 CFU/ml measurements. One covariate was potentially significant (P<0.05), cardiothoracic/vascular procedures. Logistic regression analysis was then used to determine whether ST 5 remained associated with greater desiccation tolerance as defined as the top quartile of day 2 CFU/mL measurements, while adjusting for the covariate.

ST associated with desiccation tolerance was then examined for an association with transmission. Chi squared tests were used to examine the potential association of each of the covariates mentioned above with transmission, except that age was tested Using t-test. Poisson regression with robust variance was then used to estimate the incidence risk ratios (IRR) of any transmission event for the independent variable of ST associated with desiccation tolerance after correction, ST 5, while adjusting for covariate(s) with P≤0.05 in the chi-square and t-test (age) analyses, namely general abdominal surgery. Poisson regression was used. to estimate the risk ratio because the incidence of transmission (3 3. 5% of N=173 (isolates⋅ potentially linked to intraoperative transmission) was so large that the odds ratio estimated using logistic regression would be-a biased estimator of the relative risk.

The association of transmitted ST 5 isolates with the mecA resistance trait and methicillin resistance was examined using logistic regression analysis while adjusting for covariate(s) with P≤0.05 in the chi-square or t-test (age) analyses, specifically site 0, site 2, general abdominal surgery, plastics/breast surgery, and inpatient origin.

Calculations were performed using Stata. All P-values and confidence intervals (CI) were 2-sided. No additional power analyses were conducted for this study, as all 173 S. aureus isolates were included in the analysis.

### Results

The top quartile of desiccation tolerant isolates was comprised of five clonal complexes and 7 distinct isolates that involved a total of 12 ST. ST 5, 15, 30, and 105 accounted for 67% (31/46) of desiccation tolerant ST (Table 6).

S. aureus ST type 5 isolates had greater desiccation tolerance (day 2 CFU/mL) as compared to all other ST [(MLST 5, N=34, median day 2 CFU survival 0.027%±0.029%), (other MLST, N=139, median day 2 CFU survival 0.0091% 1.41%), corrected P=0.0001]. ST 5 isolates that were transmitted had greater desiccation tolerance as compared to all other ST [(ST 5, N=15, median day 2 CFU survival 0.023%±0.037%), (other ST, N=158, median day 2 CFU survival 0.0099%±0.132%), P=0.022). ST 5 remained associated with the upper quartile of desiccation tolerance after adjusting for cardiothoracic/vascular procedures (OR 4.22, 95% CI 1.91-9.31, P=0.0004).

ST 5 was isolated from all three sites [35.29% (12/34 site 0, 11.76% (4/34) site 1, and 52.94% (18/34) site 2)] and was associated with increased risk of clonally-related transmission (RR 1.57, 95% CI 1′0.005-2.46, p=0.047) (Table 7).

OR PathTrac mapping for transmission of desiccation tolerant S. aureus isolates in the upper quartile of desiccation tolerance was employed. Provider hands and patient skin surfaces were proven sources of within and between-case transmission that led to infection. Reservoir involvement, transmission pathways, and strength of transmission links were included. The automated interpretation of the graphical display with failure mode analysis and affected OR care environments were graphed by site. T

Transmitted ST 5 isolates were associated with the mecA resistance trait (OR.adj 14.81, 95% CI 3.83-57.19, P=0.0001) and methicillin-resistance (OR.adj 4.25, 95% CI 1.29-13.98, P=0.020).

### Discussion

Desiccation tolerance is known to impact S. aureus survival, transmission, and infection development. In this study, S. aureus MLST 5 was strongly associated with increased desiccation tolerance and clonal transmission as compared to all other intraoperative MLST. Desiccation tolerant MLST 5 was also directly linked by genome analysis to postoperative infection development. As patient skin surfaces and provider hands before care were identified as important sources of intraoperative contamination with this strain characteristic, improved intraoperative hand hygiene and patient decolonization efforts may serve to attenuate transmission of this important strain characteristic.

Desiccation is a physiological process by which a substantial fraction of cellular water is removed, leading to shrinkage in cell capsular layers, a relative increase in the intracellular concentration of salt and macromolecules, and increased cellular susceptibility to oxygen free-radicals and associated damage to phospholipids, DNA, and proteins. As this process disrupts cellular function and inhibits proliferation, the ability to survive desiccation and/or to proliferate despite desiccation may result in environmental persistence and transmission.

This work shows that S. aureus ST 5 isolates have a greater strength of association with desiccation tolerance than other intraoperative sequence types isolated in this study. In turn, ST 5 isolates are more likely to be transmitted and were associated with the spread of mecA and methicillin resistance. Finally, desiccation tolerant isolates were linked by whole cell genome analysis to the causative organisms of postoperative infection. Thus, this work links desiccation tolerance to transmission, spread of resistance, and infection development among the ST 5 isolates in this study. These results are consistent with the association of MLST 5 with USA100, a common cause of invasive, hospital-acquired infections. Thus, desiccation tolerance may in part explain the global success of USA 100 and warrants further consideration for the implementation and evaluation of improved infection control efforts targeting this important strain characteristic.

Provider hands and patient skin surfaces were identified as important intraoperative reservoirs, indicating that a multimodal program may serve as a best practice for infection control improvement initiatives. These findings are in alignment with recent work showing that even 100% hand hygiene compliance is not enough to prevent the spread of MRSA, a strain characteristic often associated with MLST 5. The association of hospital origin with methicillin resistance among transmitted ST 5 isolates may explain why S. aureus decolonization efforts have been particularly effective when applied to patients exposed to the hospital arena as compared to less effective implementation strategies involving only elective patients. Therefore, this work provides a rationale for extending decolonization to hospitalized patients. As the OR PathTrac surveillance technology used in this study was able to map transmission of more tolerant isolates to specific operating environments, future infection control efforts could also address operating room exposure via targeted used of include improved routine and terminal cleaning procedures.

Our study was limited by the insensitivity of the culture methods employed that may under estimate the true magnitude of the problem. In addition, our assessments were limited by the MLST isolates obtained from operating rooms in this study. However, we were able to isolate S. aureus ST 5 isolates from all three hospital centers, and study results parallel those of prior work as described above, indicating that the association of this sequence type with increased desiccation tolerance is not an isolated issue.

In conclusion, intraoperative S. aureus ST 5 is associated with increased desiccation tolerance that is in turn associated with increased risk of clonal transmission, spread of methicillin resistance, and postoperative infection development. Future work should examine the impact of improved infection control measures targeting this key strain characteristic. Patient decolonization and hand hygiene improvements are reasonable first steps.

TABLE 6
Intraoperative S. aureus Isolates with Greater Desiccation
Tolerance Stratified by Site and Sequence Type (ST)

| ST | Site 0 (N* = 64) | Site 1 (N = 37) | Site 2 (N = 64) | Total N (%) | |
|---|---|---|---|---|---|
| 5 | 4 | 0 | 14 | 18 | (42.86) |
| 105 | 5 | 0 | 0 | 5 | (11.90) |
| 15 | 2 | 1 | 1 | 4 | (9.52) |
| 30 | 1 | 3 | 0 | 4 | (9.52) |
| 8 | 0 | 0 | 2 | 1 | (4.76) |
| 59 | 0 | 0 | 2 | 2 | (4.76) |
| 72 | 1 | 0 | 1 | 2 | (4.76) |
| 1 | 0 | 0 | 1 | 1 | (2.38) |
| 20 | 1 | 0 | 0 | 1 | (2.38) |
| 50 | 1 | 0 | 0 | 1 | (2.38) |
| 188 | 1 | 0 | 0 | 1 | (2.38) |
| 1049 | 0 | 0 | 1 | 1 | (2.38) |

*N = total cases observed at a given site

TABLE 7
ST 5 and the Risk of Clonally-Related Transmission

| Clonally-Related Transmission | RR* | 95% Cl | P-Value |
|---|---|---|---|
| ST 5 | 1.81 | 1.22-2.69 | 0.003 |
| General abdominal surgery | 0.48 | 0.24-0.96 | 0.038 |

*Incidence rate ratio,
Cl = Confidence Interval,
ST = sequence type

### REFERENCES

Archer et al., Virulence, 2:445 (2011).
Bode et al., N. Engl. J. Med., 362:9 (2010).
Boucher et al., Clin. Infect. Dis., 48:1 (2009).
Boucher et al., Clinical Infectious Diseases, 48:1 (2009).
Burgess et al., Int. J. Food Microbiol., 221:37 (2016).
Caffau & Nadali, Friuli Med., 20:515 (1965).
Carrel et al., Emerg. Infect. Dis., 21:1973 (2015).
Centers for Disease Control and Prevention. Updated guidelines for evaluating public health surveillance systems: recommendations from the guidelines working group. MMWR 2001; 50:1-467 35.
Climo et al., N. Engl. J. Med., 368:533 (2013).
Dellinger & Gordon, Special Report, Anesthesiology, General Surgery, and OB/GYN News, McMahon Publishing Group. 1-10.2006.
Edmiston et al., Surgery, 138:573 (2005).
Edwards et al., Am. J. Infect. Control, 37:783 (2009).
Foweraker, J. Hosp. Infect., 29:315 (1995).
Friss & Helms, Laeger, 125:619 (1963).
Fukada et al., Masui, 45:1026 (1996).
Goldstein et al., Infect Control Hosp. Epidemiol., 38:945 (2017).
Haley et al., Am. J. Epidemiol., 111:472 (1980).
Hasman et al., J. Clin. Microbiol., 52:139 (2014).
Hedges, Int. J. Food Microbiol., 76:207 (2002).
Hekiert et al., Otolaryngol Head Neck Surg., 141:448 (2009).
http://www.whitehouse.gov/the-press-office/2014/09/18/fact-sheet-obama-administration-takes-actions-combat-antibiotic-resistan
http://www.cicsupp0li.com/cicgenomicsworkbench/801/index.php?manual=Fixed Ploidy Variant Detection.html
Jamal et al., J. Chin. Med. Assoc., S1726 (2017).
Kirkland et al., Infect Control Hosp. Epidemiol., 20:725 (1999).
Klevens et al., JAMA, 298:1763 (2007).
Koff et al., Anesthesiology, 110:978 (2009).
Koff et al., Infect Control Hosp. Epidemiol., 37:888 (2016).
Konvalinka et al., J. Hosp. Infect., 64:162 (2006).
Leung & Chan, Med. Sci. Monit., 12:p.SR1 7-23 (2006).
Lindquist K. HOW CAN I ESTIMATE RELATIVE RISK USING GLM FOR COMMON 523 OUTCOMES IN COHORT STUDIES?. https://stats.idre.ucla.edu/stata/faq/how-can-i-estimate-relative-risk-using-glm-for-common-outcomes-in-coh0lt-studies/. Accessed Sep. 9, 2017.
Loftus et al., Am. J. Infect Control, 2018 Jan. 4. pii: S0196-6553(17)31224-5. doi: 10.1016/j.ajic.2017.11.002. [Epub ahead of print]
Loftus et al., Am. J. Infect. Control, (2018).
Loftus et al., Anesth. Analg., 114:1236 (2012c).
Loftus et al., Anesth. Analg., 115:1109 (2012b).
Loftus et al., Anesth. Analg., 115:1315 (2012a).
Loftus et al., Anesth. Analg., 120:807 (2015).
Loftus et al., Anesthesiology, 109:399 (2008).
Madar et al., Bratisl. Lek. Listy, 106:348 (2005).
Maslyk et al., AANA J., 70:53 (2002).
McComi et al., FEMS Microbiol. Lett., 353:157 (2014).
Miles et al., J. Hyg., 38:732 (1938).
Noto et al., J. Bacteriol., 190:1276 (2008).
Parikh et al., J. Pediatr. Orthop., 30:617 (2010).
Phillips et al., Infect Control Hosp. Epidemiol., 35:826 (2014).
Potts, Microbiol. Rev., 58:755 (1994).
Prasad et al., J. Gen. Intern. Med., 23:635 (2008).
Procopio et al., Anesth. Analg., 93:460 (2001).
Reddy & Loftus, R W. Microbial Contamination of Today's Operating Room Environments. Abstract 143:1. IA.RS 2018 Annual Meeting and International Science Symposium, Apr. 28-May 1, 2018.
Schultea et al., J. Clin. Microbiol., 51:212 (2013).
Schultea et al., J. Clin. Microbiol., 51:212 (2013).
Schweizer et al., JAMA, 313:2162 (2015).
Shamsudin et al., Infect. Genet. Evol., 18:106 (2013).
Shaw et al., BMC Infect. Dis., 18:4 (2018).
Stulberg et al., JAMA, 303:2479 (2010).
Vogel et al., Surg. Infect. (Larchmt), 13:307 (2012).
von Eiff et al., N. Engl. J. Med., 344:11 (2001).
Wielders et al., J. Clin. Microbiol., 40:3970 (2002).
World Health Organization. Antimicrobial Resistance. Global Report on Surveillance. 463 WHO. 2014
Young & O'Regan, Interact Cardiovasc. Thorne Surg., 10:526 (2010).
Zerbino et al., Genome Res., 18:821 (2008).
Zou, American Journal of Epidemiology, 159:702 (2004).

The inventions being thus described, it will be obvious that the same may be varied in many ways. Such variations are not to be regarded as a departure from the spirit and scope of the inventions and all such modifications are intended to be included within the scope of the following claims. The above specification provides a description of the manufacture and use of the disclosed compositions and methods. Since many embodiments can be made without departing from the spirit and scope of the invention, the invention resides in the claims.

What is claimed is:

1. An allelic discrimination panel comprising:
a multilocus sequence type (MLST) assay, wherein the MLST assay detects a bacterial genus, bacterial species, a bacterial strain, or a combination thereof.

2. The allelic discrimination panel of claim 1, wherein the MLST assay comprises a polymerase chain reaction (PCR) platform, a PCR seal, a PCR, a primer, a PCR reagent, a probe, or combination thereof.

3. The allelic discrimination panel of claim 2, wherein the PCR is a real-time PCR.

4. The allelic discrimination panel of claim 1, wherein the MLST assay is an S. aureus assay, which detects an S. aureus strain.

5. The allelic discrimination panel of claim 1, wherein the MLST assay is an MLST 5, MLST 8, MLST 15, MLST 30, or MLST 59 assay of S. aureus.

6. The allelic discrimination panel of claim 1, wherein the MLST assay targets a position in a bacteria genome, a position in a bacterial plasmid, or a combination thereof.

7. The allelic discrimination panel of claim 6, wherein the position in the bacteria genome and/or the position in the bacterial plasmid is one or more of the following 189723, 716410, 1517201, 1517381, 64695, 64954, 64974, 65001, 65096, 65216, 66001, 66200, 66543, 105870, 657832, 727126, 908100, 1186436, 1419668, 1656006, 1762538, 1897263, 2209374, 2211044, or 2257298.

8. The allelic discrimination panel of claim 1, further comprising an additional assay; wherein the additional assay detects a bacterial genus and/or species.

9. The allelic discrimination panel of claim 1, further comprising a second MLST assay, wherein the second MLST assay detects an additional sequence type.

10. The allelic discrimination panel of claim 1, wherein the probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a combination thereof.

11. A method of identifying and/or monitoring a pathogenic bacterial strain among commonly isolated intraoperative multilocus sequence types comprising: performing an MLST assay comprising detecting the presence of an MLST of a bacteria, wherein the detecting is performed by a real-time PCR comprising a probe.

12. The method of claim 11, wherein the MLST assay is an S. aureus MLST assay.

13. The method of claim 11, wherein the MLST assay is an MLST 5, MLST 8, MLST 15, MLST 30, or MLST 59 assay of S. aureus.

14. The method of claim 11, further comprising performing a second MLST assay, wherein the second MLST assay detects an additional sequence type.

15. The method of claim 11, wherein the MLST assay PCR probe comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a combination thereof.

16. The method of claim 11, wherein the MLST assay targets a position in a bacteria genome, a position in a bacterial plasmid, or a combination thereof.

17. The method of claim 16, wherein the position in the bacteria genome and/or the position in the bacterial plasmid is one or more of the following 189723, 716410, 1517201, 1517381, 64695, 64954, 64974, 65001, 65096, 65216, 66001, 66200, 66543, 105870, 657832, 727126, 908100, 1186436, 1419668, 1656006, 1762538, 1897263, 2209374, 2211044, or 2257298.

18. A kit for identifying and/or monitoring pathogenic bacterial strains among commonly isolated intraoperative multilocus sequence types comprising: the allelic discrimination panel of claim 1.

19. The kit of claim 18, further comprising instructions, a swab, a buffer, a glove, or a combination thereof.


SEQUENCE LISTING (78 sequences)

SEQ ID NO: 1 - DNA, 17 bp, Staphylococcus aureus
aacattgtagcgcctaa

SEQ ID NO: 2 - DNA, 18 bp, Staphylococcus aureus
aaacattgtaccgcctaa

SEQ ID NO: 3 - DNA, 20 bp, Staphylococcus aureus
aagaaagaaaacaagcgcta

SEQ ID NO: 4 - DNA, 20 bp, Staphylococcus aureus
aagaaagaaaacaagcgtta

SEQ ID NO: 5 - DNA, 15 bp, Staphylococcus aureus
tgttcaacggctgct

SEQ ID NO: 6 - DNA, 16 bp, Staphylococcus aureus
tgttcaacggctactt

SEQ ID NO: 7 - DNA, 22 bp, Staphylococcus aureus
tgtatttaattcagatgccgtt

SEQ ID NO: 8 - DNA, 20 bp, Staphylococcus aureus
atttaattcagatgccattg

SEQ ID NO: 9 - DNA, 19 bp, Staphylococcus aureus
tacgtttgaactctgcgac

SEQ ID NO: 10 - DNA, 17 bp, Staphylococcus aureus
cgtttgaactctgcaac

SEQ ID NO: 11 - DNA, 25 bp, Staphylococcus aureus
ccttcaagtttagcactacgacctt

SEQ ID NO: 12 - DNA, 28 bp, Staphylococcus aureus
gcaaacggacataaagtagtaactgaaa

SEQ ID NO: 13 - DNA, 17 bp, Staphylococcus aureus
aacattgtagcgcctaa

SEQ ID NO: 14 - DNA, 18 bp, Staphylococcus aureus
aaacattgtaccgcctaa

SEQ ID NO: 15 - DNA, 27 bp, Staphylococcus aureus
gagccatacttcgacaaactaacataa

SEQ ID NO: 16 - DNA, 23 bp, Staphylococcus aureus
tgcgcctatgacattgattaatg

SEQ ID NO: 17 - DNA, 20 bp, Staphylococcus aureus
aagaaagaaaacaagcgcta

SEQ ID NO: 18 - DNA, 20 bp, Staphylococcus aureus
aagaaagaaaacaagcgtta

SEQ ID NO: 19 - DNA, 25 bp, Staphylococcus aureus
caaaacaccagtgactgctatgaaa

SEQ ID NO: 20 - DNA, 26 bp, Staphylococcus aureus
aacatcgagttaatacgtgaccattc

SEQ ID NO: 21 - DNA, 15 bp, Staphylococcus aureus
tgttcaacggctgct

SEQ ID NO: 22 - DNA, 16 bp, Staphylococcus aureus
tgttcaacggctactt

SEQ ID NO: 23 - DNA, 19 bp, Staphylococcus aureus
tttaatcgcgcgtcaccat

SEQ ID NO: 24 - DNA, 22 bp, Staphylococcus aureus
aacagctggcacaaatgacaat

SEQ ID NO: 25 - DNA, 22 bp, Staphylococcus aureus
tgtatttaattcagatgccgtt

SEQ ID NO: 26 - DNA, 20 bp, Staphylococcus aureus
atttaattcagatgccattg

SEQ ID NO: 27 - DNA, 27 bp, Staphylococcus aureus
tgtagctgcttcatcattaataccatt

SEQ ID NO: 28 - DNA, 25 bp, Staphylococcus aureus
aacagttgcaggtgtaaatcaagtg

SEQ ID NO: 29 - DNA, 20 bp, Staphylococcus aureus
gggacccgcacaagcggtgg

SEQ ID NO: 30 - DNA, 20 bp, Staphylococcus aureus
gggttgcgctcgttgcggga

SEQ ID NO: 31 - DNA, 20 bp, Staphylococcus aureus
gaggtaaagccaacgcactc

SEQ ID NO: 32 - DNA, 20 bp, Staphylococcus aureus
cctgtaaccgcaccaagttt

SEQ ID NO: 33 - DNA, 20 bp, Staphylococcus aureus
ataccggcgactgggtttat

SEQ ID NO: 34 - DNA, 21 bp, Staphylococcus aureus
ttgcaaatcgtgggtatgtgt

SEQ ID NO: 35 - DNA, 20 bp, Staphylococcus aureus
cttgggtatttgcacgcatt

SEQ ID NO: 36 - DNA, 20 bp, Staphylococcus aureus
gcaatatcatgccgacacct

SEQ ID NO: 37 - DNA, 20 bp, Staphylococcus aureus
acccaacgctaaaatcatcg

SEQ ID NO: 38 - DNA, 20 bp, Staphylococcus aureus
gcgaaaatgcccatagtttc

SEQ ID NO: 39 - DNA, 24 bp, Staphylococcus aureus
acccaggttcagattctggcagcg

SEQ ID NO: 40 - DNA, 23 bp, Staphylococcus aureus
tcgctgagtcggaatcgcttgct

SEQ ID NO: 41 - DNA, 20 bp, Staphylococcus aureus
aactccagggccgccggttg

SEQ ID NO: 42 - DNA, 24 bp, Staphylococcus aureus
cctgagtcgctgtctgagcctgag

SEQ ID NO: 43 - DNA, 23 bp, Staphylococcus aureus
ggtgcagctggtgcaatgggtgt

SEQ ID NO: 44 - DNA, 21 bp, Staphylococcus aureus
gctgcgcctccagccaaacct

SEQ ID NO: 45 - DNA, 20 bp, Staphylococcus aureus
aaattgggagcagcatcagt

SEQ ID NO: 46 - DNA, 20 bp, Staphylococcus aureus
gcagctgaattcccattttc

SEQ ID NO: 47 - DNA, 21 bp, Staphylococcus aureus
acgctcaaggcgacggcaaag

SEQ ID NO: 48 - DNA, 25 bp, Staphylococcus aureus
accttctgcatgaccttctgcacct

SEQ ID NO: 49 - DNA, 21 bp, Staphylococcus aureus
cgtcaacagcagatgcgagcg

SEQ ID NO: 50 - DNA, 24 bp, Staphylococcus aureus
tgcatcagttttcgctgctggttt

SEQ ID NO: 51 - DNA, 23 bp, Staphylococcus aureus
tgccgtaggtgacgaaggtggtt

SEQ ID NO: 52 - DNA, 22 bp, Staphylococcus aureus
gcaccgtgttcgccttcgaact

SEQ ID NO: 53 - DNA, 20 bp, Staphylococcus aureus
aatagaggcgccacgaccgt

SEQ ID NO: 54 - DNA, 24 bp, Staphylococcus aureus
gtgccttcccaaaccttttgagca

SEQ ID NO: 55 - DNA, 61 bp, Staphylococcus aureus
catttttaaattttacagacactgtttccggtataatgaaattaatttgaaaattcaggat

SEQ ID NO: 56 - DNA, 61 bp, Staphylococcus aureus
catttttaaattttacagacactgtttccgctataatgaaattaatttgaaaattcaggat

SEQ ID NO: 57 - DNA, 61 bp, Staphylococcus aureus
gagcttaaggaacaagggaagattaaagccgttggtgtatcaaatttcacattagatcaac

SEQ ID NO: 58 - DNA, 61 bp, Staphylococcus aureus
gagcttaaggaacaagggaagattaaagccattggtgtatcaaatttcacattagatcaac

SEQ ID NO: 59 - DNA, 61 bp, Staphylococcus aureus
aagaattaagagataaatatgaatggaacctcagtttactctctaaaattgacaactacct

SEQ ID NO: 60 - DNA, 61 bp, Staphylococcus aureus
aagaattaagagataaatatgaatggaaccccagtttactctctaaaattgacaactacct

SEQ ID NO: 61 - DNA, 61 bp, Staphylococcus aureus
tcaaagatgtaactacattttatgaggaaggtaaacatttaatctatggttatacaccaac

SEQ ID NO: 62 - DNA, 61 bp, Staphylococcus aureus
tcaaagatgtaactacattttatgaggaagataaacatttaatctatggttatacaccaac

SEQ ID NO: 63 - DNA, 61 bp, Staphylococcus aureus
tttctgcatgtcgaggatttttaacataactgtttgtgtcagttagttttaactttttact

SEQ ID NO: 64 - DNA, 61 bp, Staphylococcus aureus
tttctgcatgtcgaggatttttaacataacggtttgtgtcagttagttttaactttttact

SEQ ID NO: 65 - DNA, 61 bp, Staphylococcus aureus
caacctagacgtgaatggattgaaaagcattttgagtttggtatgcaagaggaccaaagta

SEQ ID NO: 66 - DNA, 61 bp, Staphylococcus aureus
caacctagacgtgaatggattgaaaagcatgttgagtttggtatgcaagaggaccaaagta

SEQ ID NO: 67 - DNA, 61 bp, Staphylococcus aureus
tcttccccgacccagtcatcagcatcatcagggcttacaccattcgcttcaccaacagtca

SEQ ID NO: 68 - DNA, 60 bp, Staphylococcus aureus
tcttccccgacccagtcatcagcatcatcaggcttacaccattcgcttcaccaacagtca

SEQ ID NO: 69 - DNA, 61 bp, Staphylococcus aureus
atagtcacagcttctttaaaatgagttatgacttcatcaatattctcatttttcataaata

SEQ ID NO: 70 - DNA, 61 bp, Staphylococcus aureus
atagtcacagcttctttaaaatgagttatggcttcatcaatattctcatttttcataaata

SEQ ID NO: 71 - DNA, 61 bp, Staphylococcus aureus
ttatctgtttctgcttgcgcttctttcttcacttcttgaatcgcttgtgcttcttgtgatg

SEQ ID NO: 72 - DNA, 61 bp, Staphylococcus aureus
ttatctgtttctgcttgcgcttctttcttcgcttcttgaatcgcttgtgcttcttgtgatg

SEQ ID NO: 73 - DNA, 61 bp, Staphylococcus aureus
caaatccaaaacaatttgacgtcatcgtttacgaaaacttatttggcgatattttaagtga

SEQ ID NO: 74 - DNA, 61 bp, Staphylococcus aureus
caaatccaaaacaatttgacgtcatcgtttgcgaaaacttatttggcgatattttaagtga

SEQ ID NO: 75 - DNA, 60 bp, Staphylococcus aureus
gcaagaacacatttagtatcccctgctatggcagcagcagctattcatggtaaatttgtg

SEQ ID NO: 76 - DNA, 63 bp, Staphylococcus aureus
gcaagaacacatttagtatcccctgctatggcagcagcagcagctattcatggtaaatttgtg

SEQ ID NO: 77 - DNA, 61 bp, Staphylococcus aureus
gatattagcattcatacgtttgaactctgcgacatgcataaaacgattttcaaatacagtt

SEQ ID NO: 78 - DNA, 61 bp, Staphylococcus aureus
gatattagcattcatacgtttgaactctgcaacatgcataaaacgattttcaaatacagtt